(19) United States
(12) Patent Application Publication
(10) Pub. No.: US 2022/0040169 A1
(43) Pub. Date: Feb. 10, 2022

(54) Methods for the Administration of Certain VMAT2 Inhibitors

(71) Applicant: Neurocrine Biosciences, Inc., San Diego, CA (US)

(72) Inventors: Grace S. Liang, San Diego, CA (US); Eiry W. Roberts, San Diego, CA (US); Dao Tuyet Thai-Cuarto, San Diego, CA (US); Barbara Scholz, Del Mar, CA (US)

(21) Appl. No.: 17/506,883

(22) Filed: Oct. 21, 2021

Related U.S. Application Data
(62) Division of application No. 17/007,710, filed on Aug. 31, 2020.
Application No. 17/007,710 is a continuation of application No. PCT/US2020/003218, filed on May 8, 2020.
(60) Provisional application No. 62/845,599, filed on May 9, 2019.

(51) Int. Cl. A61K 31/4745; A61P 25/16

(52) U.S. Cl. CPC A61K 31/4745; A61P 25/16

(57) ABSTRACT

Provided is a method of administering a vesicular monoamine transport 2 (VMAT2) inhibitor to a patient in need thereof, wherein the patient experiences one or more clinically significant parkinson-like signs or symptoms.

Dysregulation of dopaminergic systems is integral to several central nervous system (CNS) disorders, including neurological and psychiatric diseases and disorders. These neurological and psychiatric diseases and disorders include hyperkinetic movement disorders, and conditions such as schizophrenia and mood disorders. The transporter protein vesicular monoamine transporter-2 (VMAT2) plays an important role in presynaptic dopamine release and regulates monoamine uptake from the cytoplasm to the synaptic vesicle for storage and release.

Despite the advances that have been made in this field, there remains a need for new therapeutic products useful to treatment of neurological and psychiatric diseases and disorders and other related diseases or conditions described herein. One such agent is valbenazine, which has the following chemical structure:

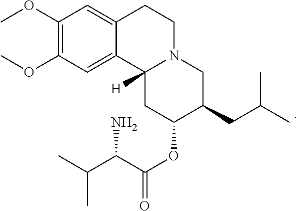 [SMILES: COc1cc2c(cc1OC)[C@H]1C[C@@H](OC(=O)[C@@H](N)C(C)C)[C@H](CC(C)C)CN1CC2]

A formulation of the VMAT2 inhibitor, valbenazine:4-toluenesulfonate (1:2) (referred to herein as “valbenazine ditosylate” or “ditosylate salt of valbenazine”) has been previously reported in the FDA approved drug label INGREZZA® (hereafter referred to as INGREZZA). INGREZZA was approved in the United States on 11 Apr. 2017 for the treatment of adults with tardive dyskinesia (TD).

There is a significant, unmet need for methods of administering a VMAT2 inhibitor, such as valbenazine, or a pharmaceutically acceptable salt and/or isotopic variant thereof, to a patient in need thereof with reduced adverse events, such as parkinsonism. The present disclosure fulfills these and other needs, as evident in reference to the following disclosure.

### SUMMARY

Provided is a method of administering a vesicular monoamine transport 2 (VMAT2) inhibitor to a patient in need thereof, comprising:

administering a therapeutically effective amount of the VMAT2 inhibitor to the patient;

monitoring the patient for one or more clinically significant parkinson-like signs or symptoms; and

administering a reduced amount of the VMAT2 inhibitor to the patient, if the patient experiences one or more clinically significant parkinson-like signs or symptoms,

wherein if the VMAT2 inhibitor is tetrabenazine or deutrabenazine, the VMAT2 inhibitor is being administered to treat a disease or disorder other than Huntington's Disease.

Also provided is a method of administering a vesicular monoamine transport 2 (VMAT2) inhibitor to a patient in need thereof, wherein the patient is experiencing one or more clinically significant parkinson-like signs or symptoms, comprising administering a reduced amount of the VMAT2 inhibitor to the patient, wherein the reduced amount of the VMAT2 administered is less than the therapeutically effective amount that would be administered to a patient who is not experiencing one or more clinically significant parkinson-like signs or symptoms, wherein if the VMAT2 inhibitor is tetrabenazine or deutrabenazine, the VMAT2 inhibitor is being administered to treat a disease or disorder other than Huntington's Disease.

In some embodiments, the method further comprising discontinuing the administration of the VMAT2 inhibitor based on the patient's ability to tolerate one or more clinically significant parkinson-like signs and symptoms.

Also provided is a method of administering a vesicular monoamine transport 2 (VMAT2) inhibitor to a patient in need thereof, comprising:

administering a therapeutically effective amount of the VMAT2 inhibitor to the patient;

monitoring the patient for one or more clinically significant parkinson-like signs or symptoms; and

discontinuing the administration of the VMAT2 inhibitor to the patient if the patient experiences one or more clinically significant parkinson-like signs or symptoms,

wherein if the VMAT2 inhibitor is tetrabenazine or deutrabenazine, the VMAT2 inhibitor is being administered to treat a disease or disorder other than Huntington's Disease.

In some embodiments, the VMAT2 inhibitor is chosen from valbenazine, or a pharmaceutically acceptable salt and/or isotopic variant thereof. In some embodiments, the VMAT2 inhibitor is valbenazine, or a pharmaceutically acceptable salt thereof. In some embodiments, the VMAT2 inhibitor is a valbenazine tosylate salt. In some embodiments, the VMAT2 inhibitor is a valbenazine tosylate salt. In some embodiments, the VMAT2 inhibitor is a ditosylate salt of valbenazine. In some further embodiments, the ditosylate salt of valbenazine is substantially crystalline.

In some embodiments, the VMAT2 inhibitor is an isotopic variant that is L-Valine, (2R,3R,11bR)-1,3,4,6,7,11b-hexahydro-9,10-di(methoxy-d3)-3-(2-methylpropyl)-2H-benzo[a]quinolizin-2-yl ester or a pharmaceutically acceptable salt thereof.

In some embodiments, the VMAT2 inhibitor is tetrabenazine (9,10-dimethoxy-3-isobutyl-1,3,4,6,7,11b-hexahydro-2H-pyrido[2,1-a]isoquinolin-2-one), or a pharmaceutically acceptable salt and/or isotopic variant thereof. In some embodiments, tetrabenazine is chosen from the RR, SS, RS, and SR isomers of tetrabenazine, and mixtures thereof. In some embodiments, tetrabenazine is a mixture of the RR and SS isomers.

In some embodiments, the VMAT2 inhibitor is deutetrabenazine.

In some embodiments, the VMAT2 inhibitor is chosen from dihydrotetrabenazine (2-hydroxy-3-(2-methylpropyl)-1,3,4,6,7,11b-hexahydro-9,10-dimethoxy-benzo(a)quinolizine), or a pharmaceutically acceptable salt and/or isotopic variant thereof. In some embodiments, dihydrotetrabenazine is chosen from the RRR, SSS, SSRR, RSS, SSR, RRS, RSR, and SRS isomers of dihydrotetrabenazine, and mixtures thereof. In some embodiments, the VMAT2 inhibitor is the RRR isomer ((+)-α-3-isobutyl-9,10-dimethoxy-1,3,4,6,7,11b-hexahydro-2H-pyrido[2,1-a]isoquinolin-2-ol), or a pharmaceutically acceptable salt and/or isotopic variant thereof.

Also provided is a method of treating a patient with a hyperkinetic movement disorder, such as tardive dyskinesia, comprising:

orally administering to the patient for a period of up to two weeks a therapeutically effective amount of a vesicular monoamine transport 2 (VMAT2) inhibitor chosen from valbenazine and pharmaceutically acceptable salts thereof; and

if the patient does not experience one or more clinically significant parkinson-like signs or symptoms during the period of up to two weeks, continuing administration of the VMAT2 inhibitor at the therapeutically effective dose or initiating administration of the VMAT2 inhibitor at an increased dose.

Also provided is a method of treating a patient with a hyperkinetic movement disorder, such as tardive dyskinesia, comprising:

orally administering to the patient for a period of up to two weeks a therapeutically effective amount of a vesicular monoamine transport 2 (VMAT2) inhibitor chosen from valbenazine and pharmaceutically acceptable salts thereof; and

if the patient experiences one or more clinically significant parkinson-like signs or symptoms during the period of up to two weeks, discontinuing administration of the VMAT2 inhibitor at the therapeutically effective dose or initiating administration of the VMAT2 inhibitor at a reduced dose.

In some embodiments, the method further comprises selecting a patient for continued treatment with the VMAT2 inhibitor if the patient has not experienced one or more clinically significant parkinson-like signs or symptoms within the first two weeks after starting or increasing the dose of the VMAT2 inhibitor administered to the patient.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, “valbenazine” or “valbenazine free base” may be referred to as (S)-2-amino-3-methyl-butyric acid (2R, 3R,11bR)-3-isobutyl-9,10-dimethoxy-1,3,4,6,7,11b-hexahydro-2H-pyrido[2,1-a]isoquinolin-2-yl ester; or as L-Valine, (2R,3R,11bR)-1,3,4,6,7,11b-hexahydro-9,10-dimethoxy-3-(2-methylpropyl)-2H-benzo[a]quinolizin-2-yl ester or as NBI-98854 with the following structure:

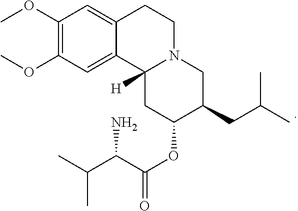 [SMILES: COc1cc2c(cc1OC)[C@H]1C[C@@H](OC(=O)[C@@H](N)C(C)C)[C@H](CC(C)C)CN1CC2]

INGREZZA contains valbenazine, present as valbenazine ditosylate salt, with the chemical name, L-Valine, (2R,3R,11bR)-1,3,4,6,7,11b-hexahydro-9,10-dimethoxy-3-(2-methylpropyl)-2H-benzo[a]quinolizin-2-yl ester, 4-methylbenzenesulfonate (1:2). Valbenazine ditosylate is slightly soluble in water. Its molecular formula is C38H54N2O10S2, and its molecular weight is 762.97/mol (ditosylate salt) with the following structure:

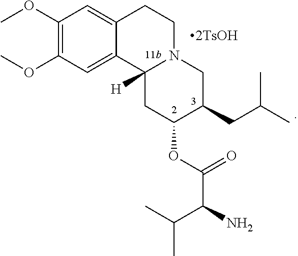 [SMILES: COc1cc2c(cc1OC)[C@H]1C[C@@H](OC(=O)[C@@H](N)C(C)C)[C@H](CC(C)C)CN1CC2]

Valbenazine ditosylate may exist as an amorphous form and crystalline Forms I-VI. The synthesis and characterization of the amorphous form and crystalline Forms I-VI of valbenzine ditosylate have been described in the U.S. Pat. No. 10,065,952, which is incorporated herein by reference in its entirety for all purposes.

As used herein, “tetrabenazine” may be referred to as 1,3,4,6,7,11b-hexahydro-9,1 O-dimethoxy-3-(2-methylpropyl)-2H-benzo(a)quinolizin-2-one. The compound has chiral centers at the 3 and 11b carbon atoms and hence can, theoretically, exist in a total of four isomeric forms as shown below:

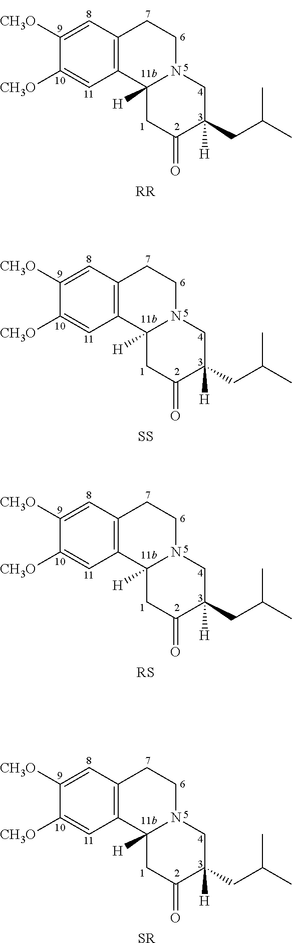

Commercially available tetrabenazine is a racemic mixture of the RR and SS isomers. See, e.g., XENAZINE (tetrabenazine) US Prescribing Information, Sep. 13, 2017, which is incorporated herein by reference in its entirety for all purposes.

As used herein, “deutetrabenazine” may be referred to as (RR, SS)-1,3,4,6,7,11b-hexahydro-9,10-di(methoxyd3)-3-(2-methylpropyl)-2H-benzo[a]quinolizin-2-one. Deutetrabenazine is a racemic mixture containing the following compounds:

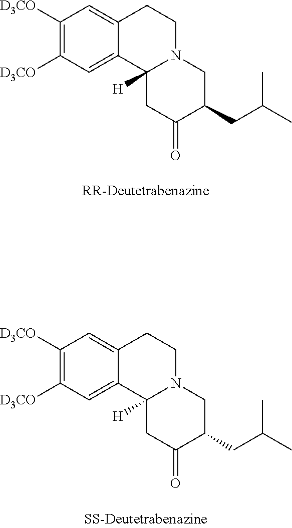

See, e.g., AUSTEDO (deutetrabenazine) US Prescribing Information, Jun. 6, 2018, which is incorporated herein by reference in its entirety for all purposes.

As used herein, “dihydrotetrabenazine” may be referred to as 2-hydroxy-3-(2-methylpropyl)-1,3,4,6,7,11b-hexahydro-9,10-dimethoxy-benzo(a)quinolizine. The compound has three chiral centers and hence can, theoretically, exist in a total of eight isomeric forms as shown below:

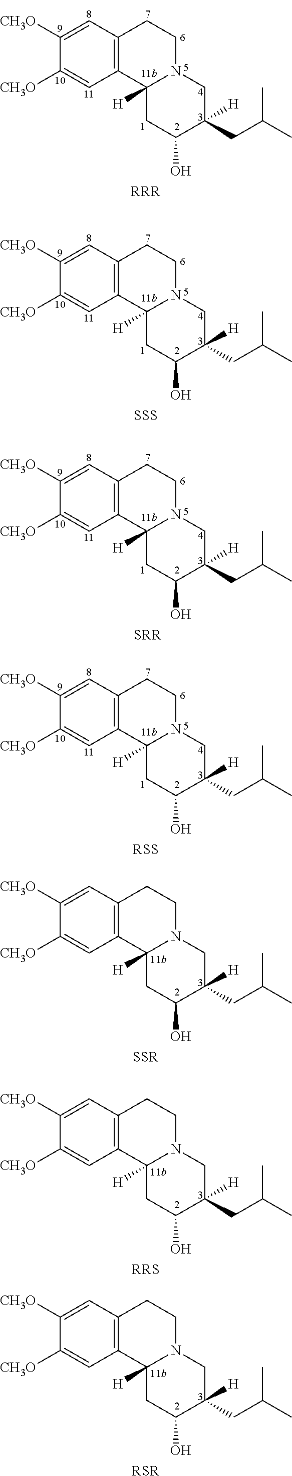

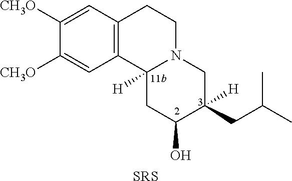

The synthesis and characterization of the eight isomers is described by Sun et al. (2011) Eur. J. Med. Chem. 1841-1848, which is incorporated herein by reference in its entirety for all purposes.

As used herein, “isotopic variant” means a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (H), deuterium (2H), tritium (H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (2H), carbon-12 (¹²C), carbon-13 (¹³C), nitrogen-14 (14N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound contains unnatural proportions of one or more isotopes, including, but not limited to, tritium (3H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a compound contains an unnatural proportion of deuterium.

With regard to the compounds provided herein, when a particular atomic position is designated as having deuterium or “D” or “d”, it is understood that the abundance of deuterium at that position is substantially greater than the natural abundance of deuterium, which is about 0.015%. A position designated as having deuterium typically has a minimum isotopic enrichment factor of, in certain embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position. The isotopic enrichment of the compounds provided herein can be determined using conventional analytical methods known to one of ordinary skill in the art, including mass spectrometry, nuclear magnetic resonance spectroscopy, and crystallography.

As used herein, “about” means ±20% of the stated value, and includes more specifically values of ±10%, ±5%, ±2% and ±1% of the stated value.

As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

As used herein, “adjusting administration”, “altering administration”, “adjusting dosing”, or “altering dosing” are all equivalent and mean tapering off, reducing or increasing the dose of the substance, ceasing to administer the substance to the patient, or substituting a different active agent for the substance.

As used herein, “administering to a patient” refers to the process of introducing a composition or dosage form into the patient via an art-recognized means of introduction.

As used herein, “clinically stable” means the patient is in a state of health or disease from which little if any immediate change is expected. For example, a patient is considered clinically stable if the patient has been on a consistent dosage of medication for at least one month. Clinically stable patients may be symptomatic; however, the symptoms should be at a consistent level in terms of type and severity.

As used herein, “clinically significant” refers to a change in a subject's clinical condition, such as a level of a side effect, that a physician treating the subject would consider to be important.

As used herein, a “dose” means the measured quantity of an active agent to be taken at one time by a patient. In certain embodiments, wherein the active agent is not valbenazine free base, the quantity is the molar equivalent to the corresponding amount of valbenazine free base. For example, often a drug is packaged in a pharmaceutically acceptable salt form, for example valbenazine ditosylate, and the dosage for strength refers to the mass of the molar equivalent of the corresponding free base, valbenazine. As an example, 73 mg of valbenazine ditosylate is the molar equivalent of 40 mg of valbenazine free base.

As used herein, “dosing regimen” means the dose of an active agent taken at a first time by a patient and the interval (time or symptomatic) at which any subsequent doses of the active agent are taken by the patient such as from about 20 to about 160 mg once daily, e.g., about 20, about 40, about 60, about 80, about 100, about 120, or about 160 mg once daily. The additional doses of the active agent can be different from the dose taken at the first time.

As used herein, “effective amount” and “therapeutically effective amount” of an agent, compound, drug, composition or combination is an amount which is nontoxic, tolerable, and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, “a medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, “Medication Guide” means an FDA-approved patient labeling for a pharmaceutical product conforming to the specifications set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, “parkinson-like signs or symptoms” or “parkinsonism” is a general term that refers to a group of neurological conditions or disorders related to motor function similar to those seen in Parkinson's disease but that may be caused by a condition other than Parkinson's disease. The Simpson-Angus Scale (SAS) can be utilized to evaluate for parkinsonism. See, Simpson et al. (1970) Acta Psychiatry Scand Suppl 212:11-19. This scale contains 10 items: gait, arm dropping, shoulder shaking, elbow rigidity, wrist rigidity, leg pendulousness, head dropping, glabella tap, tremor, and salivation. Each item is rated between 0 and 4. A total score is obtained by adding the items and dividing by 10. Scores of up to 0.3 are considered within the normal range.

As used herein, “patient” or “individual” or “subject” means a mammal, including a human, for whom or which therapy is desired, and generally refers to the recipient of the therapy.

As used herein, “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree.

As used herein, “pharmaceutically acceptable salt” means any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) salts formed when an acidic proton present in the parent compound either (a) is replaced by a metal ion, e.g., an alkali metal ion, an alkaline earth ion or an aluminum ion, or alkali metal or alkaline earth metal hydroxides, such as sodium, potassium, calcium, magnesium, aluminum, lithium, zinc, and barium hydroxide, ammonia, or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like. Pharmaceutically acceptable salts further include, by way of example only and without limitation, sodium, potassium, calcium, magnesium, ammonium, tetraalkylammonium, and the like, and when the compound contains a basic functionality, salts of non-toxic organic or inorganic acids, such as hydrohalides, e.g. hydrochloride and hydrobromide, sulfate, phosphate, sulfamate, nitrate, acetate, trifluoroacetate, trichloroacetate, propionate, hexanoate, cyclopentylpropionate, glycolate, glutarate, pyruvate, lactate, malonate, succinate, sorbate, ascorbate, malate, maleate, fumarate, tartarate, citrate, benzoate, 3-(4-hydroxybenzoyl)benzoate, picrate, cinnamate, mandelate, phthalate, laurate, methanesulfonate (mesylate), ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluconate, benzoate, glutamate, hydroxynaphthoate, salicylate, stearate, cyclohexylsulfamate, quinate, muconate, and the like.

As used herein, “crystalline” refers to a solid in which the constituent atoms, molecules, or ions are packed in a regularly ordered, repeating three-dimensional pattern. In particular, a crystalline compound or salt might be produced as one or more crystalline forms. Different crystalline forms may be distinguished by X-ray powder diffraction (XRPD) patterns.

As used herein, “substantially crystalline” refers to compounds or salts that are at least a particular weight percent crystalline. In some embodiments, the compound or salt is substantially crystalline. Examples of a crystalline form or substantially crystalline form include a single crystalline form or a mixture of different crystalline forms. Particular weight percentages include 50%, 60%, 70%, 75%, 80%, 85%, 87%, 88%, 89%, 90%, 91%, 92%, 93%, 94%, 95%, 96%, 97%, 98%, 99%, 99.5% and 99.9%. In some embodiments, substantially crystalline refers to compounds or salts that are at least 70% crystalline. In some embodiments, substantially crystalline refers to compounds or salts that are at least 80% crystalline. In some embodiments, substantially crystalline refers to compounds or salts that are at least 85% crystalline. In some embodiments, substantially crystalline refers to compounds or salts that are at least 90% crystalline. In some embodiments, substantially crystalline refers to compounds or salts that are at least 95% crystalline. In some embodiments, substantially crystalline refers to compounds or salts that are at least 98% crystalline. In some embodiments, substantially crystalline refers to compounds or salts that are at least 99% crystalline.

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “treating” or “treatment” refers to therapeutic applications to slow or stop progression of a disorder, prophylactic application to prevent development of a disorder, and/or reversal of a disorder. Reversal of a disorder differs from a therapeutic application which slows or stops a disorder in that with a method of reversing, not only is progression of a disorder completely stopped, cellular behavior is moved to some degree, toward a normal state that would be observed in the absence of the disorder.

As used herein, “VMAT2” refers to human vesicular monoamine transporter isoform 2, an integral membrane protein that acts to transport monoamines, particularly neurotransmitters such as dopamine, norepinephrine, serotonin, and histamine, from cellular cytosol into synaptic vesicles.

As used herein, the term “VMAT2 inhibitor”, “inhibit VMAT2”, or “inhibition of VMAT2” refers to the ability of a compound disclosed herein to alter the function of VMAT2. A VMAT2 inhibitor may block or reduce the activity of VMAT2 by forming a reversible or irreversible covalent bond between the inhibitor and VMAT2 or through formation of a noncovalently bound complex. Such inhibition may be manifest only in particular cell types or may be contingent on a particular biological event. The term “VMAT2 inhibitor”, “inhibit VMAT2”, or “inhibition of VMAT2” also refers to altering the function of VMAT2 by decreasing the probability that a complex forms between a VMAT2 and a natural substrate.

As used herein, “experience” has the same meaning as “develop” and “occur”.

As used herein “hypersensitivity” or “hypersensitivity reaction” refers to an immunological sensitization due to a drug and/or its metabolites. Generally, there are four types of hypersensitivity:

Type I, IgE mediated—immediate-type hypersensitivity, including systemic hypersensitivity (e.g., anaphylaxis and urticarial) and respiratory hypersensitivity (e.g., asthma);

Type II, IgG or IgM mediated—antibody-mediated cytotoxic reaction and Type III, IgG mediated—immune complex reaction, which often occur simultaneously and are commonly associated with systemic or organ hypersensitivity reactions. Type II and III immunopathies include anemia, leukopenia, thrombocytopenia, pneumonitis, vasculitis, lupus-like reactions or glomeronephritis; and

Type IV, T lymphocyte mediated—delayed-type hypersensitivity response, which most commonly occurs as a delayed-type hypersensitivity skin reaction.

Provided herein is a method of administering a vesicular monoamine transport 2 (VMAT2) inhibitor chosen from valbenazine and pharmaceutically acceptable salts and/or isotopic variants thereof, to a patient in need thereof, wherein the patient is experiencing one or more clinically significant parkinson-like signs or symptoms, comprising: discontinuing administration of the VMAT2 inhibitor.

Also provided is a method of administering a vesicular monoamine transport 2 (VMAT2) inhibitor chosen from valbenazine and pharmaceutically acceptable salts and/or isotopic variants thereof, to a patient in need thereof, wherein the patient is experiencing one or more clinically significant parkinson-like signs or symptoms, comprising: administrating a reduced amount of the VMAT2 inhibitor, wherein the reduced amount is less than a therapeutically effective amount that would be administered to a patient who is not experiencing one or more clinically significant parkinson-like signs or symptoms.

Also provided is a method of administering a vesicular monoamine transport 2 (VMAT2) inhibitor chosen from valbenazine and pharmaceutically acceptable salts and/or isotopic variants thereof, to a patient in need thereof, comprising: administering a therapeutically effective amount of the VMAT2 inhibitor to the patient; monitoring the patient for one or more clinically significant parkinson-like signs or symptoms; and discontinuing administration of the VMAT2 inhibitor to the patient.

Also provided is a method of administering a vesicular monoamine transport 2 (VMAT2) inhibitor chosen from valbenazine and pharmaceutically acceptable salts and/or isotopic variants thereof, to a patient in need thereof, comprising: administering a therapeutically effective amount of the VMAT2 inhibitor to the patient; monitoring the patient for one or more clinically significant parkinson-like signs or symptoms; and administering a reduced amount of the VMAT2 inhibitor to the patient.

Also provided is a method of administering a vesicular monoamine transport 2 (VMAT2) inhibitor to a patient in need thereof, comprising administering a first therapeutically effective amount of the VMAT2 inhibitor to the patient; monitoring the patient for one or more clinically significant parkinson-like signs or symptoms; and administering a second therapeutically effective amount of the VMAT2 inhibitor to the patient if the patient is experiencing one or more clinically significant parkinson-like signs or symptoms, and wherein if the VMAT2 inhibitor is tetrabenazine or deutrabenazine, the VMAT2 inhibitor is being administered to treat a disease or disorder other than Huntington's Disease.

Also provided is a method of administering a vesicular monoamine transport 2 (VMAT2) inhibitor to a patient in need thereof, comprising; administering a therapeutically effective amount of the VMAT2 inhibitor to the patient; monitoring the patient for one or more clinically significant parkinson-like signs or symptoms; and administering the same therapeutically effective amount or an increased amount of the VMAT2 inhibitor to the patient if the patient is not experiencing one or more clinically significant parkinson-like signs or symptoms, and wherein if the VMAT2 inhibitor is tetrabenazine or deutrabenazine, the VMAT2 inhibitor is being administered to treat a disease or disorder other than Huntington's Disease.

In some embodiments, the dose in increased by at least 10%, at least 20%, at least 30%, at least 40% at least 50%, or at least 60% from the therapeutically effective amount. In some embodiments, the therapeutically effective amount is about 40 mg of valbenazine free base once daily, the increased amount is about 80 mg of valbenazine free base once daily. In some embodiments, the therapeutically effective amount is about 60 mg of valbenazine free base once daily, the increased amount is about 80 mg of valbenazine free base once daily. In some embodiments, the therapeutically effective amount is about 60 mg of valbenazine free base once daily, the increased amount is about 80 mg of valbenazine free base once daily.

Also provided is a method of administering a vesicular monoamine transport 2 (VMAT2) inhibitor to a patient in need thereof, comprising: administering a first therapeutically effective amount of the VMAT2 inhibitor to the patient; monitoring the patient for one or more clinically significant parkinson-like signs or symptoms; and administering a second therapeutically effective amount of the VMAT2 inhibitor to the patient if the patient is not experiencing one or more clinically significant parkinson-like signs or symptoms, wherein if the VMAT2 inhibitor is tetrabenazine or deutrabenazine, the VMAT2 inhibitor is being administered to treat a disease or disorder other than Huntington's Disease.

Also provided is a method of treating a patient with a neurological or psychiatric disease or disorder, comprising: administering a therapeutically effective amount of a vesicular monoamine transport 2 (VMAT2) inhibitor to the patient; monitoring the patient for one or more clinically significant parkinson-like signs or symptoms; and adjusting the amount of the VMAT2 inhibitor to the patient, if the patient experiences one or more clinically significant parkinson-like signs or symptoms, wherein if the VMAT2 inhibitor is tetrabenazine or deutrabenazine, the VMAT2 inhibitor is being administered to treat a disease or disorder other than Huntington's Disease.

In some embodiment, the adjusting the amount is discontinuing the treatment. In some embodiments, the adjusting the amount is administering a reduced amount.

Also provided is a method of treating a patient with hyperkinetic movement disorder, comprising: orally administering an effective amount of a vesicular monoamine transport 2 (VMAT2) inhibitor chosen from valbenazine and pharmaceutically acceptable salts and/or isotopic variants thereof to the patent; monitoring the patient for one or more clinically significant parkinson-like signs or symptoms; and administering a reduced amount of the VMAT2 inhibitor to the patient if the patient experiences one or more clinically significant parkinson-like signs or symptoms.

Also provide is a method of treating a patient with hyperkinetic movement disorder, comprising: orally administering to the patient an effective amount of a vesicular monoamine transport 2 (VMAT2) inhibitor chosen from valbenazine and pharmaceutically acceptable salts and/or isotopic variants thereof; monitoring the patient for one or more clinically significant parkinson-like signs or symptoms; and discontinuing the treatment of the VMAT2 inhibitor to the patient, if the patient experiences one or more clinically significant parkinson-like signs or symptoms.

Also provided is a method of treating a patient with a neurological or psychiatric disease or disorder, comprising: administering to the patient a reduced amount of a vesicular monoamine transport 2 (VMAT2) inhibitor chosen from valbenazine and pharmaceutically acceptable salts and/or isotopic variants thereof, wherein the reduced amount of the VMAT2 administered is less than the therapeutically effective amount that would be administered to a patient who is not experiencing one or more clinically significant parkinson-like signs or symptoms.

Also provided is a method of treating a patient with hyperkinetic movement disorder, comprising: orally administering to the patient a therapeutically effective amount of a vesicular monoamine transport 2 (VMAT2) inhibitor chosen from valbenazine and pharmaceutically acceptable salts thereof; monitoring the patient for one or more clinically significant parkinson-like signs or symptoms; and administering a reduced amount or discontinuing the treatment of the VMAT2 inhibitor to the patient, if the patient experiences one or more clinically significant parkinson-like signs or symptoms.

Also provided is a method of treating a patient with a hyperkinetic movement disorder, such as tardive dyskinesia, comprising:

orally administering to the patient for a period of up to two weeks a therapeutically effective amount of a vesicular monoamine transport 2 (VMAT2) inhibitor chosen from valbenazine and pharmaceutically acceptable salts thereof; and

if the patient does not experience one or more clinically significant parkinson-like signs or symptoms during the period of up to two weeks, continuing administration of the VMAT2 inhibitor at the same therapeutically effective dose or initiating administration of the VMAT2 inhibitor at an increased dose.

In some embodiments, the dose in increased by at least 10%, at least 20%, at least 30%, at least 40% at least 50%, or at least 60% from the therapeutically effective amount. In some embodiments, the therapeutically effective amount is about 40 mg of valbenazine free base once daily, the increased amount is about 80 mg of valbenazine free base once daily. In some embodiments, the therapeutically effective amount is about 60 mg of valbenazine free base once daily, the increased amount is about 80 mg of valbenazine free base once daily. In some embodiments, the therapeutically effective amount is about 60 mg of valbenazine free base once daily, the increased amount is about 80 mg of valbenazine free base once daily.

Also provided is a method of treating a patient with a hyperkinetic movement disorder, such as tardive dyskinesia, comprising:

orally administering to the patient for a period of up to two weeks a therapeutically effective amount of a vesicular monoamine transport 2 (VMAT2) inhibitor chosen from valbenazine and pharmaceutically acceptable salts thereof; and

if the patient experiences one or more clinically significant parkinson-like signs or symptoms during the period of up to two weeks, administering a reduced amount of the VMAT2 inhibitor to the patient.

Also provided is a method of treating a patient with a hyperkinetic movement disorder, such as tardive dyskinesia, comprising:

orally administering to the patient for a period of up to two weeks a therapeutically effective amount of a vesicular monoamine transport 2 (VMAT2) inhibitor chosen from valbenazine and pharmaceutically acceptable salts thereof; and

if the patient experiences one or more clinically significant parkinson-like signs or symptoms during the period of up to two weeks, discontinuing administration of the VMAT2 inhibitor.

Also provided is a use of a vesicular monoamine transport 2 (VMAT2) inhibitor in the preparation of a medicament for treating a disease or disorder in a patient, wherein the medicament comprise a therapeutically effective amount of the VMAT2 inhibitor; the patient is monitored for one or more clinically significant parkinson-like signs or symptoms; the amount of the VMAT2 inhibitor administered to the patient is reduced or the administration of the VMAT 2 inhibitor is discontinued if the patient experiences one or more clinically significant parkinson-like signs or symptoms, and if the VMAT2 inhibitor is tetrabenazine or deutrabenazine, the disease or disorder is not Huntington's Disease.

Also provided is a use of a vesicular monoamine transport 2 (VMAT2) inhibitor chosen from valbenazine and a pharmaceutically acceptable salts and/or isotopic variants thereof in the preparation of a medicament for treating a disease or disorder in a patient, wherein the medicament comprise a therapeutically effective amount of the VMAT2 inhibitor; the patient is monitored for one or more clinically significant parkinson-like signs or symptoms; the amount of the VMAT2 inhibitor administered to the patient is reduced or the administration of the VMAT2 inhibitor is discontinued if the patient experiences one or more clinically significant parkinson-like signs or symptoms.

Also provided is a use of a vesicular monoamine transport 2 (VMAT2) inhibitor chosen from valbenazine and a pharmaceutically acceptable salts and/or isotopic variants thereof in the preparation of a medicament for treating a disease or disorder in a patient, wherein the medicament comprise a therapeutically effective amount of the VMAT2 inhibitor; the patient is monitored for one or more clinically significant parkinson-like signs or symptoms; if the patient does not experience one or more clinically significant parkinson-like signs or symptoms during the period of up to two weeks, continuing administration of the VMAT2 inhibitor at the same therapeutically effective dose or initiating administration of the VMAT2 inhibitor at an increased dose.

Also provided is a use of a vesicular monoamine transport 2 (VMAT2) inhibitor chosen from valbenazine and a pharmaceutically acceptable salts and/or isotopic variants thereof in the preparation of a medicament for treating a disease or disorder in a patient, wherein the medicament comprise a therapeutically effective amount of the VMAT2 inhibitor; the patient is monitored for one or more clinically significant parkinson-like signs or symptoms; if the patient experiences one or more clinically significant parkinson-like signs or symptoms during the period of up to two weeks, discontinuing administration of the VMAT2 inhibitor.

Also provided is a use of a vesicular monoamine transport 2 (VMAT2) inhibitor chosen from valbenazine and a pharmaceutically acceptable salts and/or isotopic variants thereof in the preparation of a medicament for treating a disease or disorder in a patient, wherein the medicament comprise a therapeutically effective amount of the VMAT2 inhibitor; the patient is monitored for one or more clinically significant parkinson-like signs or symptoms; if the patient experiences one or more clinically significant parkinson-like signs or symptoms during the period of up to two weeks, administering the VMAT2 inhibitor at a reduced amount.

In some embodiments, the patient is an adult.

In some embodiments, the method or use further comprises discontinuing administration of the VMAT2 inhibitor based on the patient's ability to tolerate one or more clinically significant parkinson-like signs or symptoms after a reduced amount or dose of the VMAT2 inhibitor is administered. In some embodiments, the administration is discontinued for a first period of time, such as at least one week, e.g., one, two, three or four weeks, and then administration is continued at a reduced dose.

In some embodiments, the method or use further comprises informing the patient or a medical care worker that administration of the VMAT2 inhibitor to the patient may result in one or more clinically significant parkinson-like signs or symptoms. In some embodiments, the method further comprises informing the patient or a medical care worker that administration of the VMAT2 inhibitor to the patient may result in increased risk of the one or more clinically significant parkinson-like signs or symptoms. In some embodiments, the method or use further comprises informing the patient or a medical care worker that administration of the VMAT2 inhibitor to the patient may result in worsening of pre-existing parkinsonism or parkinson-like signs or symptoms. In some embodiments, the method or use further comprises informing the patient to report to a medical care worker any clinically significant parkinson-like signs or symptoms.

In some embodiments, the VMAT2 inhibitor is not administered to the patient having pre-existing parkinsonism. In some embodiments, the method or use further comprises determining whether the patient has pre-existing parkinsonism prior to initiation of treatment with the VMAT2 inhibitor.

In some embodiments, the method or use further comprises administering to the patient that is experiencing one or more clinically significant parkinson-like signs or symptoms one or more medications used to treat Parkinson disease. In some embodiments, the medication is a dopamine decarboxylase inhibitor in combination with a dopamine precursor, e.g., the dopamine decarboxylase inhibitor carbidopa or benserazide in combination with the dopamine precursor levodopa. In some embodiments, the medication is a catechol-o-methyltransferase (COMT) inhibitor, such as entacapone, tolcapone, or opicapone. In some embodiments, the medication is a dopamine decarboxylase inhibitor in combination with a dopamine precursor in further combination with a COMT inhibitor. In some embodiments, the medication is a dopamine agonist, such as pramipexole, ropinirole, apomorphine, bromocriptine, or rotigotine. In some embodiments, the medication is a monoamine oxidase B inhibitor such as selegiline, rasagiline, or safinamide. In some embodiments, the medication is amantadine. In some embodiments, the medication is an anticholinergic drug, such as trihexyphenidyl or benztropine. In some embodiments, the medication is chosen from levodopa, carbidopa, and opicapone. In some embodiments, the medication is chosen from levodopa, carbidopa, and entacapone. In some embodiments, the medication is levodopa in combination with carbidopa and opicapone. In some embodiments, the medication is chosen from rivastigmine, cariprazine, and paliperidone.

In some embodiments, prior to administration of the therapeutically effective amount of the VMAT2 inhibitor, the patient had a Simpson-Angus Scale score of <0.3. In some embodiments, after administration of the therapeutically effective amount of the VMAT2 inhibitor, the patient had a change in Simpson-Angus Scale score of >1.

In some embodiments, prior to the administration, the patient is at increased risk of experiencing one or more clinically significant parkinson-like signs or symptoms.

In some embodiments, the patient at increased risk of experiencing clinically significant parkinson-like signs or symptoms is a patient who is being co-administered one or more antipsychotics, antidepressants, antiepileptics, or other drugs that are known to possibly cause parkinsonism. In some embodiments, the patient is being co-administered one or more drugs chosen from amlodipine, atropine, benztropine, clonazepam, clozapine, fluoxetine, gabapentin, Lamictal, lisinopril, lithium, lurasidone, olanzapine, oxycodone, paliperidone, pregabalin, prazosin, quetiapine, tiotixene, tizanidine, valproic acid, and valproate. In some embodiments, the patient being co-administered one or more other drugs is clinically stable.

In some embodiments, the patient at increased risk of experiencing clinically significant parkinson-like signs or symptoms is a patient who is being co-administered one or more antipsychotics. In some embodiments, the antipsychotic drug is a typical antipsychotic drug. In some embodiments, the typical antipsychotic drug is fluphenazine, haloperidol, loxapine, molindone, perphenazine, pimozide, sulpiride, thioridazine, or trifluoperazine. In some embodiments, the antipsychotic drug is an atypical antipsychotic drug. In some embodiments, the atypical antipsychotic drug is aripiprazole, asenapine, clozapine, iloperidone, olanzapine, paliperidone, quetiapine, risperidone, or ziprasidone. In some embodiments, the atypical antipsychotic drug is clozapine.

In some embodiments, the patient at increased risk of experiencing clinically significant parkinson-like signs or symptoms is a patient having pre-existing parkinsonism. In some embodiments, the patient at increased risk of experiencing clinically significant parkinson-like signs or symptoms is a patient having pre-existing Parkinson's disease (also referred to as idiopathic Parkinson's disease.) In some embodiments, the patient at increased risk of experiencing clinically significant parkinson-like signs or symptoms is a patient having a pre-existing condition chosen from corticobasal degeneration, dementia with Lewy Bodies, drug-induced parkinsonism, essential tremor, multiple system atrophy, progressive supranuclear palsy, and vascular parkinsonism. In some embodiments, the patient at increased risk of experiencing clinically significant parkinson-like signs or symptoms is a patient having a pre-existing gait disorders. In some embodiments, the patient having a pre-existing condition is clinically stable.

In some embodiments, prior to administration of the therapeutically effective amount of the VMAT2 inhibitor, the patient had a Simpson-Angus Scale score of ≥0.3. In some embodiments, after administration of the therapeutically effective amount of the VMAT2 inhibitor, the patient had a change in Simpson-Angus Scale score of >1.

In some embodiments, the one or more clinically significant parkinson-like signs or symptoms is chosen from difficulty moving or loss of ability to move muscles voluntarily, tremor, gait disturbances and drooling. In some embodiments, the one or more clinically significant parkinson-like signs or symptoms is chosen from akinesia, severe tremor, gait disturbances (shuffling, festination) and drooling. In some embodiments, the one or more clinically significant parkinson-like signs or symptoms is chosen from falls, gait disturbances, tremor, drooling and hypokinesia. In some embodiments, the one or more clinically significant parkinson-like signs or symptoms is chosen from shaking, body stiffness, trouble moving or walking and trouble keeping balance.

In some embodiments, the one or more clinically significant parkinson-like signs or symptoms occurs within the first two weeks after starting or increasing the dose of the VMAT2 inhibitor administered to the patient.

In some embodiments, the method further comprises selecting a patient for continued treatment with the VMAT2 inhibitor if the patient has not experienced one or more clinically significant parkinson-like signs or symptoms within the first two weeks after starting or increasing the dose of the VMAT2 inhibitor administered to the patient.

In some embodiments, the one or more clinically significant parkinson-like signs or symptoms occurs within the first two weeks of administration of the VMAT2 inhibitor. In some embodiments, the one or more clinically significant parkinson-like signs or symptoms occurs within the first two weeks of increasing the amount of the VMAT2 inhibitor administered to the patient.

In some embodiments, the severity of at least one of the one or more clinically significant parkinson-like signs or symptoms is reduced after discontinuing administration of the VMAT2 inhibitor. In some embodiments, at least one of the one or more clinically significant parkinson-like signs or symptoms is resolved after discontinuing administration of the VMAT2 inhibitor. In a further embodiment, the VMAT2 inhibitor is valbenazine ditosylate.

In some embodiments, the VMAT2 inhibitor is chosen from valbenazine and pharmaceutically acceptable salts and/or isotopic variants thereof. In some embodiments, the VMAT2 inhibitor is valbenazine, or a pharmaceutically acceptable salt thereof. In some embodiments, the VMAT2 inhibitor is a valbenazine salt. In some embodiments, the VMAT2 inhibitor is a valbenazine tosylate salt. In some embodiments, the VMAT2 inhibitor is a ditosylate salt of valbenazine. In some embodiments, the ditosylate salt of valbenazine is amorphous.

In some embodiments, the ditosylate salt of valbenazine is substantially amorphous. In some embodiments, the ditosylate salt of valbenazine is substantially crystalline. In some embodiments, the crystalline ditosylate salt of valbenazine has an XRPD diffraction pattern comprising X-ray diffraction peaks at two-theta angles of 6.3, 17.9, and 19.7°±0.2°. In some embodiments, the crystalline ditosylate salt of valbenazine has an XRPD diffraction pattern comprising X-ray diffraction peaks at two-theta angles of 6.3 and 17.9°±0.2°. In some embodiments, the crystalline ditosylate salt of valbenazine has an XRPD diffraction pattern comprising X-ray diffraction peaks at two-theta angle of 6.3°±0.2°. In some embodiments, crystalline Form I has a DSC thermogram comprising an endothermic event with an onset temperature of about 240° C. and a peak at about 243° C.

In some embodiments, the VMAT2 inhibitor is chosen from dihydrotetrabenazine (2-hydroxy-3-(2-methylpropyl)-1,3,4,6,7,11b-hexahydro-9,10-dimethoxy-benzo(a)quinolizine) and a pharmaceutically acceptable salt and/or isotopic variant thereof. In some embodiments, dihydrotetrabenazine is chosen from the RRR, SSS, SSRR, RSS, SSR, RRS, RSR, and SRS isomers of dihydrotetrabenazine, and mixtures thereof. In some embodiments, the VMAT2 inhibitor is the RRR isomer ((+)-α-3-isobutyl-9,10-dimethoxy-1,3,4,6,7,11b-hexahydro-2H-pyrido[2,1-a]isoquinolin-2-ol), or a pharmaceutically acceptable salt and/or isotopic variant thereof.

In some embodiments, the VMAT2 inhibitor is administered orally. In some embodiments, the VMAT2 inhibitor is administered in the form of a tablet or capsule.

In some embodiments, the VMAT2 inhibitor is administered with or without food.

In some embodiments, the VMAT2 inhibitor is administered in an amount equivalent to between about 20 mg and about 160 mg, between about 20 mg and about 150 mg, between 20 mg and about 140 mg, between about 20 mg and about of 130 mg, between about 20 mg and 120 mg, between about 30 mg and 120 mg, or between about 40 mg and about 120 mg once daily. In some embodiments, the VMAT2 inhibitor is administered in an amount equivalent to between about 20 mg and about 100 mg once daily. In some embodiments, the VMAT2 inhibitor is administered in an amount equivalent to about 20 mg once daily. In some embodiments, the VMAT2 inhibitor is administered in an amount equivalent to about 40 mg once daily. In some embodiments, the VMAT2 inhibitor is administered in an amount equivalent to about 50 mg once daily. In some embodiments, the VMAT2 inhibitor is administered in an amount equivalent to about 60 mg once daily. In some embodiments, the VMAT2 inhibitor is administered in an amount equivalent to about 70 mg once daily. In some embodiments, the VMAT2 inhibitor is administered in an amount equivalent to about 80 mg once daily. In some embodiments, the VMAT2 inhibitor is administered in an amount equivalent to about 100 mg once daily. In some embodiments, the VMAT2 inhibitor is administered in an amount equivalent to about 120 mg once daily.

In some embodiments, the VMAT2 inhibitor is administered in an amount equivalent to between about 20 mg and about 160 mg, between about 20 mg and about 150 mg, between 20 mg and about 140 mg, between about 20 mg and about of 130 mg, between about 20 mg and 120 mg, between about 30 mg and 120 mg, or between about 40 mg and about 120 mg. In some embodiments, the VMAT2 inhibitor is administered in an amount equivalent to between about 20 mg and about 100 mg. In some embodiments, the VMAT2 inhibitor is administered in an amount equivalent to about 20 mg of. In some embodiments, the VMAT2 inhibitor is administered in an amount equivalent to about 40 mg. In some embodiments, the VMAT2 inhibitor is administered in an amount equivalent to about 50 mg. In some embodiments, the VMAT2 inhibitor is administered in an amount equivalent to about 60 mg. In some embodiments, the VMAT2 inhibitor is administered in an amount equivalent to about 70 mg. In some embodiments, the VMAT2 inhibitor is administered in an amount equivalent to about 80 mg. In some embodiments, the VMAT2 inhibitor is administered in an amount equivalent to about 100 mg. In some embodiments, the VMAT2 inhibitor is administered in an amount equivalent to about 120 mg.

In some embodiments, the therapeutically effective amount is an amount equivalent to between about 20 mg and about 160 mg, between about 20 mg and about 150 mg, between 20 mg and about 140 mg, between about 20 mg and about of 130 mg, between about 20 mg and 120 mg, between about 20 mg and 110 mg, between about 20 mg and 100 mg, between about 30 mg and 120 mg, or between about 40 mg and about 120 mg valbenazine free base once daily. In some embodiments, the therapeutically effective amount is an amount equivalent to between about 20 mg and about 100 mg of valbenazine free base once daily. In some embodiments, the therapeutically effective amount is an amount equivalent to between about 20 mg and about 120 mg of valbenazine free base once daily. In some embodiments, the therapeutically effective amount is an amount equivalent to about 20 mg of valbenazine free base once daily. In some embodiments, the therapeutically effective amount is an amount equivalent to about 40 mg of valbenazine free base once daily. In some embodiments, the therapeutically effective amount is an amount equivalent to about 50 mg of valbenazine free base once daily. In some embodiments, the therapeutically effective amount is an amount equivalent to about 60 mg of valbenazine free base once daily. In some embodiments, the therapeutically effective amount is an amount equivalent to about 70 mg of valbenazine free base once daily. In some embodiments, the therapeutically effective amount is an amount equivalent to about 80 mg of valbenazine free base once daily. In some embodiments, the therapeutically effective amount is an amount equivalent to about 100 mg of valbenazine free base once daily. In some embodiments, the therapeutically effective amount is an amount equivalent to about 120 mg of valbenazine free base once daily.

In some embodiments, the therapeutically effective amount is an amount equivalent to between about 20 mg and about 160 mg, between about 20 mg and about 150 mg, between 20 mg and about 140 mg, between about 20 mg and about of 130 mg, between about 20 mg and 120 mg, between about 20 mg and 110 mg, between about 20 mg and 100 mg, between about 30 mg and 120 mg, or between about 40 mg and about 120 mg valbenazine free base. In some embodiments, the therapeutically effective amount is an amount equivalent to between about 20 mg and about 100 mg of valbenazine free base. In some embodiments, the therapeutically effective amount an amount equivalent to about 20 mg of valbenazine free base. In some embodiments, the therapeutically effective amount is an amount equivalent to about 40 mg of valbenazine free base. In some embodiments, the therapeutically effective amount is an amount equivalent to about 50 mg of valbenazine free base. In some embodiments, the therapeutically effective amount is an amount equivalent to about 60 mg of valbenazine free base. In some embodiments, the therapeutically effective amount is an amount equivalent to about 70 mg of valbenazine free base. In some embodiments, the therapeutically effective amount is an amount equivalent to about 80 mg of valbenazine free base once. In some embodiments, the therapeutically effective amount is an amount equivalent to about 100 mg of valbenazine free base. In some embodiments, the therapeutically effective amount is an amount equivalent to about 120 mg of valbenazine free base.

In some embodiments, the reduced amount of the VMAT2 inhibitor is reduced by at least about 10% of a therapeutically effective amount that would be administered to a patient who does not experience one or more clinically significant parkinson-like signs or symptoms as a result of administration of the VMAT2 inhibitor. In some embodiments, the reduced amount of the VMAT2 inhibitor is reduced by at least about 20% of the therapeutically effective amount. In some embodiments, the reduced amount of the VMAT2 inhibitor is reduced by at least about 30% of the therapeutically effective amount. In some embodiments, the reduced amount of the VMAT2 inhibitor is reduced by at least about 40% of the therapeutically effective amount. In some embodiments, the reduced amount of the VMAT2 inhibitor is reduced by at least about 50% of the therapeutically effective amount. In some embodiments, the reduced amount of the VMAT2 inhibitor is reduced by at least about 60% of the therapeutically effective amount. In some embodiments, the reduced amount of the VMAT2 inhibitor is reduced by at least about 70% of the therapeutically effective amount. In some embodiments, the reduced amount of the VMAT2 inhibitor is reduced by at least about 80% of the therapeutically effective amount. In some embodiments, the reduced amount of the VMAT2 inhibitor is reduced by at least about 90% of the therapeutically effective amount.

In certain embodiments, the reduced amount of the VMAT2 inhibitor is 10-90% less than the amount that would be administered to a patient who does not experience one or more clinically significant parkinson-like signs or symptoms as a result of administration of the VMAT2 inhibitor. In certain embodiments, the reduced amount of the VMAT2 inhibitor is 20-80% less than the amount that would be administered to a patient who does not experience one or more clinically significant parkinson-like signs or symptoms as a result of administration of the VMAT2 inhibitor. In certain embodiments, the reduced amount of the VMAT2 inhibitor is 30-70% less than the amount that would be administered to a patient who does not experience one or more clinically significant parkinson-like signs or symptoms as a result of administration of the VMAT2 inhibitor. In certain embodiments, the reduced amount of the VMAT2 inhibitor is 40-60% less than the amount that would be administered to a patient who does not experience one or more clinically significant parkinson-like signs or symptoms as a result of administration of the VMAT2 inhibitor. In certain embodiments, the reduced amount of the VMAT2 inhibitor is about 50% less than the amount that would be administered to a patient who does not experience one or more clinically significant parkinson-like signs or symptoms as a result of administration of the VMAT2 inhibitor. In some embodiments, the amount that would be administered to a patient who does not experience one or more clinically significant parkinson-like signs or symptoms as a result of administration of the VMAT2 inhibitor is between about 20 mg to about 120 mg per day. In some embodiments, the amount that would be administered to a patient who does not experience one or more clinically significant parkinson-like signs or symptoms as a result of administration of the VMAT2 inhibitor is about 40 mg, about 60 mg, or 80 mg once daily.

In certain embodiments, the reduced amount of the VMAT2 inhibitor is between about 10 mg to about 80 mg, between about 10 mg to about 70 mg, between about 10 mg to about 60 mg, between about 10 mg to about 50 mg, between about 10 mg to about 40 mg, between about 10 mg to about 30 mg, between about 20 mg to about 80 mg, between about 20 mg to about 70 mg, between about 20 mg to about 60 mg, between about 20 mg to about 50 mg, between about 20 mg to about 40 mg, between about 20 mg to about 30 mg, between about 30 mg to about 80 mg, between about 30 mg to about 70 mg, between about 30 mg to about 60 mg, between about 30 mg to about 50 mg, between about 30 mg to about 40 mg of valbenazine per day. In a further embodiment, the VMAT 2 inhibitor is administered once daily.

In certain embodiments, the reduced amount or dose of the VMAT2 inhibitor is about 10 mg, about 15 mg, about 20 mg, about 25 mg, about 30 mg, about 35 mg, about 40 mg, about 45 mg, about 50 mg, about 55 mg, or about 60 mg of valbenazine per day. In some embodiments, the reduced amount is about 40 mg of valbenazine per day. In some embodiments, the reduced amount is about 60 mg of valbenazine per day. In a further embodiment, the VMAT 2 inhibitor is administered once daily.

For example, wherein the amount that would be administered to a patient who does not experience one or more clinically significant parkinson-like signs or symptoms as a result of administration of the VMAT2 inhibitor is about 40 mg per day, an individual may receive a reduced dosage of about 36, 35, 32, 30, 28, 25, 24, 20, 16, 12, 8, or 4 mg per day. Likewise, wherein the amount that would be administered to a patient who does not experience one or more clinically significant parkinson-like signs or symptoms as a result of administration of the VMAT2 inhibitor is about 60 mg per day, an individual may receive a reduced dosage of about 56, 50, 48, 45, 40, 32, 30, 24, 16, or 8 per day. Likewise, wherein the amount that would be administered to a patient who does not experience one or more clinically significant parkinson-like signs or symptoms as a result of administration of the VMAT2 inhibitor is about 80 mg per day, an individual may receive a reduced dosage of about 72, 64, 60, 56, 50, 48, 45, 40, 32, 30, 24, 20, 16, or 8 per day. In a further embodiment, the VMAT2 inhibitor is administered once daily.

For example, wherein the dosage administered to a patient who does not experience one or more clinically significant parkinson-like signs or symptoms as a result of administration of the VMAT2 inhibitor is about 40 mg per day, an individual may receive a reduced dosage of about 4-36 mg per day, about 8-32 mg per day, about 12-28 mg per day, about 16-24 mg per day, or in certain embodiments, about 20 mg per day. In some embodiments, wherein the dosage administered to a patient who does not experience one or more clinically significant parkinson-like signs or symptoms as a result of administration of the VMAT2 inhibitor is about 80 mg per day, an individual may receive a reduced dosage of about 8-72 mg per day, about 16-64 mg per day, about 24-56 mg per day, about 32-48 mg per day, or in certain embodiments, about 24 mg per day. In a further embodiment, the VMAT2 inhibitor is administered once daily.

In some embodiments, wherein the dosage administered to a patient who does not experience one or more clinically significant parkinson-like signs or symptoms as a result of administration of the VMAT2 inhibitor is about 40 mg per day, an individual may receive a reduced dosage of about 5-35 mg per day, about 10-30 mg per day, about 15-30 mg per day, about 15-25 mg per day, or in certain embodiments, about 20 mg per day or about 30 mg per day. In some embodiments, wherein the dosage administered to a patient who does not experience one or more clinically significant parkinson-like signs or symptoms as a result of administration of the VMAT2 inhibitor is about 40 mg per day, an individual may receive the same dosage of about 40 mg every other day, a reduced dosage of about 5-35 mg every other day, about 10-30 mg every other day, about 15-30 mg every other day, about 15-25 mg every other day, or in certain embodiments, about 20 mg every other day or about 30 mg every other day.

In some embodiments, wherein the dosage administered to a patient who does not experience one or more clinically significant parkinson-like signs or symptoms as a result of administration of the VMAT2 inhibitor is about 60 mg per day, an individual may receive a reduced dosage of about 5-75 mg per day, about 10-70 mg per day, about 15-65 mg per day, about 20-60 mg per day, about 25-55 mg per day, about 30-50 mg per day, or in certain embodiments, about 40 mg per day. In some embodiments, wherein the dosage administered to a patient who does not experience one or more clinically significant parkinson-like signs or symptoms as a result of administration of the VMAT2 inhibitor is about 60 mg per day, an individual may receive the same dosage of about 60 mg every other day, a reduced dosage of about 5-75 mg every other day, about 10-70 mg every other day, about 15-65 mg every other day, about 20-60 mg every other day, about 25-55 mg every other day, about 30-50 mg every other day, or in certain embodiments, about 40 mg every other day.

In some embodiments, wherein the dosage administered to a patient who does not experience one or more clinically significant parkinson-like signs or symptoms as a result of administration of the VMAT2 inhibitor is about 80 mg per day, an individual may receive a reduced dosage of about 5-75 mg per day, about 10-70 mg per day, about 15-65 mg per day, about 20-60 mg per day, about 25-55 mg per day, about 30-50 mg per day, or in certain embodiments, about 40 mg per day. In some embodiments, wherein the dosage administered to a patient who does not experience one or more clinically significant parkinson-like signs or symptoms as a result of administration of the VMAT2 inhibitor is about 80 mg per day, an individual may receive the same dosage of about 80 mg every other day, a reduced dosage of about 5-75 mg every other day, about 10-70 mg every other day, about 15-65 mg every other day, about 20-60 mg every other day, about 25-55 mg every other day, about 30-50 mg every other day, or in certain embodiments, about 40 mg every other day.

In some embodiments, wherein the dosage administered to a patient who does not experience one or more clinically significant parkinson-like signs or symptoms as a result of administration of the VMAT2 inhibitor is about 80 mg per day, an individual may receive a reduced dosage of about 10-75 mg per day, about 20-70 mg per day, about 30-65 mg per day, about 40-65 mg per day, about 45-45 mg per day, or in certain embodiments, about 60 mg per day. In some embodiments, wherein the dosage administered to a patient who does not experience one or more clinically significant parkinson-like signs or symptoms as a result of administration of the VMAT2 inhibitor is about 80 mg per day, an individual may receive the same dosage of about 80 mg every other day, a reduced dosage of about 10-75 mg every other day, about 20-70 mg every other day, about 30-65 mg every other day, about 40-65 mg every other day, about 45-45 mg every other day, or in certain embodiments, about 60 mg every other day.

In some embodiments, wherein a patient who does not experience one or more clinically significant parkinson-like signs or symptoms as a result of administration of a first therapeutically effective amount of a VMAT2 inhibitor, the patient may receive a second therapeutically effective amount, wherein the second therapeutically effective amount may be the same or a higher amount compared to the first therapeutically effective amount. In some further embodiments, the first therapeutically effective amount is an amount between about 30 and 100 mg and the second therapeutically effective amount is an amount between about 40 mg and about 120 mg. In some embodiments, the first therapeutically effective amount is about 30 mg, about 40 mg, about 50 mg, about 60 mg, about 70 mg, about 80 mg, about 90 mg and about 100 mg of valbenazine free base per day. In some embodiments, the second therapeutically effective amount is about 30 mg, about 40 mg, about 50 mg, about 60 mg, about 70 mg, about 80 mg, about 90 mg, about 100 mg, about 110 mg, and about 120 mg of valbenazine free base per day. In some embodiments, the first therapeutically effective amount is about 40 mg of valbenazine free base per day, and the second therapeutically effective amount is about 40 mg of valbenazine free base per day. In some embodiments, the first therapeutically effective amount is about 40 mg of valbenazine free base per day, and the second therapeutically effective amount is about 60 mg of valbenazine free base per day. In some embodiments, the first therapeutically effective amount is about 40 mg of valbenazine free base per day, and the second therapeutically effective amount is about 80 mg of valbenazine free base per day. In some embodiments, the first therapeutically effective amount is about 60 mg of valbenazine free base per day, and the second therapeutically effective amount is about 60 mg of valbenazine free base per day. In some embodiments, the first therapeutically effective amount is about 60 mg of valbenazine free base per day, and the second therapeutically effective amount is about 80 mg of valbenazine free base per day. In some embodiments, the first therapeutically effective amount is about 80 mg of valbenazine free base per day, and the second therapeutically effective amount is about 80 mg of valbenazine free base per day.

In some embodiments, wherein a patient who experiences one or more clinically significant parkinson-like signs or symptoms as a result of administration of a first therapeutically effective amount of a VMAT2 inhibitor, the patient may receive a second therapeutically effective amount, wherein the second therapeutically effective amount is less than the amount in the first therapeutically effective amount. In some further embodiments, the first therapeutically effective amount is an amount between about 30 and 100 mg and the second therapeutically effective amount is an amount between about 40 mg and about 120 mg. In some embodiments, the first therapeutically effective amount is about 30 mg, about 40 mg, about 50 mg, about 60 mg, about 70 mg, about 80 mg, about 90 mg and about 100 mg of valbenazine free base per day. In some embodiments, the second therapeutically effective amount is about 10 mg, about 20 mg, about 30 mg, about 40 mg, about 50 mg, or about 60 mg of valbenazine free base per day. In some embodiments, the first therapeutically effective amount is about 40 mg of valbenazine free base per day, and the second therapeutically effective amount is about 30 mg of valbenazine free base per day. In some embodiments, the first therapeutically effective amount is about 60 mg of valbenazine free base per day, and the second therapeutically effective amount is about 40 mg of valbenazine free base per day. In some embodiments, the first therapeutically effective amount is about 80 mg of valbenazine free base per day, and the second therapeutically effective amount is about 40 mg of valbenazine free base per day. In some embodiments, the first therapeutically effective amount is about 80 mg of valbenazine free base per day, and the second therapeutically effective amount is about 60 mg of valbenazine free base per day. In some embodiments, the first therapeutically effective amount is about 60 mg of valbenazine free base per day, and the second therapeutically effective amount is about 30 mg of valbenazine free base per day. In some embodiments, the first therapeutically effective amount is about 60 mg of valbenazine free base per day, and the second therapeutically effective amount is about 20 mg of valbenazine free base per day.

In some embodiments, the VMAT2 inhibitor is administered in an amount sufficient to achieve a maximal blood plasma concentration (Cmax) of (+)-α-DHTBZ of between about 15 ng to about 60 ng per mL plasma and a minimal blood plasma concentration (Cmin) of approximately between about at least 33%-50% of the Cmax over a 12 hour period. In some embodiments, the VMAT2 inhibitor is administered in an amount sufficient to achieve: (i) a therapeutic concentration range of about 15 ng to about 60 ng of (+)-α-DHTBZ per mL plasma; and (ii) a threshold concentration of at least 15 ng (+)-α-DHTBZ per mL plasma over a period of about 8 hours to about 24 hours.

In some embodiments, the VMAT2 inhibitor is administered for a first period of time in a first amount and then the amount is increased to a second amount. In some embodiments, the first period of time is a week. In some embodiments, the first period of time is more than one week, such as two weeks, three weeks, or four weeks. In some embodiments, the first period of time is one month, two months, three months or more. In some embodiments, the first amount is equivalent to about 40 mg of valbenazine free base once daily. In some embodiments, the first amount is equivalent to about 60 mg of valbenazine free base once daily. In some embodiments, the second amount is equivalent to about 60 mg of valbenazine free base once daily. In some embodiments, the second amount is equivalent to about 80 mg of valbenazine free base once daily.

In some embodiments, the VMAT2 inhibitor is administered for a first period of time in a first amount and then the amount is decreased to a second amount. In some embodiments, the first period of time is a week. In some embodiments, the first period of time is more than one week, such as two weeks, three weeks, or four weeks. In some embodiments, the first period of time is one month, two months, three months or more. In some embodiments, the first amount is equivalent to about 60 mg of valbenazine free base once daily. In some embodiments, the first amount is equivalent to about 80 mg of valbenazine free base once daily. In some embodiments, the second amount is equivalent to about 40 mg of valbenazine free base once daily. In some embodiments, the second amount is equivalent to about 60 mg of valbenazine free base once daily. In some embodiments, the first amount is equivalent to about 60 mg of valbenazine free base once daily and the second amount is equivalent to about 40 mg of valbenazine free base once daily. In some embodiments, the first amount is equivalent to about 80 g of valbenazine free base once daily and the second amount is equivalent to about 40 mg of valbenazine free base once daily. In some embodiments, the first amount is equivalent to about 80 mg of valbenazine free base once daily and the second amount is equivalent to about 60 mg of valbenazine free base once daily.

In some embodiments, the VMAT2 inhibitor is administered for a first period of time in a first amount and the administration of the VMAT inhibitor is discontinued. In some embodiments, the first period of time is a week. In some embodiments, the first period of time is more than one week, such as two weeks, three weeks, or four weeks. In some embodiments, the first period of time is one month, two months, three months or more. In some embodiments, the first amount is equivalent to about 40 mg of valbenazine free base once daily. In some embodiments, the first amount is equivalent to about 60 mg of valbenazine free base once daily. In some embodiments, the first amount is equivalent to about 80 mg of valbenazine free base once daily.

In some embodiments, the VMAT2 inhibitor is administered to the patient to treat a neurological or psychiatric disease or disorder. In some embodiments, the neurological or psychiatric disease or disorder is a hyperkinetic movement disorder, mood disorder, bipolar disorder, schizophrenia, schizoaffective disorder, mania in mood disorder, depression in mood disorder, treatment-refractory obsessive compulsive disorder, neurological dysfunction associated with Lesch-Nyhan syndrome, agitation associated with Alzheimer's disease, Fragile X syndrome or Fragile X-associated tremor-ataxia syndrome, autism spectrum disorder, Rett syndrome, or chorea-acanthocytosis.

In some embodiments, the neurological or psychiatric disease or disorder is a hyperkinetic movement disorder. In some embodiments, the hyperkinetic movement disorder is tardive dyskinesia. In some embodiments, the hyperkinetic movement disorder is a tic disorder. In some embodiments, the tic disorder is Tourette's Syndrome. In some embodiments, the hyperkinetic movement disorder is Huntington's disease. In some embodiments, the hyperkinetic movement disorder is choreiform movements, general dystonia, focal dystonia, and myoclonus movements. In some embodiments, the hyperkinetic movement disorder is chorea associated with Huntington's disease. In some embodiments, the hyperkinetic movement disorder is ataxia, chorea, dystonia, Huntington's disease, myoclonus, restless leg syndrome, or tremors. In some embodiments, the hyperkinetic movement disorder is a disease or disorder other than Huntington's disease. In some embodiments, the hyperkinetic movement disorder is a disease or disorder other than Huntington's disease and the VMAT2 inhibitor is deutetrabenazine or tetrabenazine.

In some embodiments, the neurological or psychiatric disease or disorder is a hyperkinetic movement disorder in patients with intellectual and developmental disability (IDD). In some embodiments, the hyperkinetic movement disorder is tardive dyskinesia in patients with intellectual and developmental disability (IDD). In some embodiments, the hyperkinetic movement disorder is a tic disorder in patients with intellectual and developmental disability (IDD). In some embodiments, the tic disorder is Tourette's Syndrome in patients with intellectual and developmental disability (IDD). In some embodiments, the hyperkinetic movement disorder is Huntington's disease in patients with intellectual and developmental disability (IDD). In some embodiments, the hyperkinetic movement disorder is choreiform movements, general dystonia, focal dystonia, and myoclonus movements in patients with intellectual and developmental disability (IDD). In some embodiments, the hyperkinetic movement disorder is chorea associated with Huntington's disease in patients with intellectual and developmental disability (IDD). In some embodiments, the hyperkinetic movement disorder is ataxia, chorea, dystonia, Huntington's disease, myoclonus, restless leg syndrome, or tremors in patients with intellectual and developmental disability (IDD). In some embodiments, the hyperkinetic movement disorder is a disease or disorder other than Huntington's disease in patients with intellectual and developmental disability (IDD). In some embodiments, the hyperkinetic movement disorder is a disease or disorder other than Huntington's disease and the VMAT2 inhibitor is deutetrabenazine or tetrabenazine in patients with intellectual and developmental disability (IDD).

In some embodiments, the intellectual and developmental disability (IDD) comprises intellectual disability and developmental disability. In some embodiments, the intellectual and developmental disability (IDD) is intellectual disability. In some embodiments, the intellectual and developmental disability (IDD) is developmental disability. In some embodiments, the intellectual and developmental disability (IDD) is characterized by the body parts or systems being affected. In a further embodiment, the body parts or systems is selected from nervous system, sensory system, metabolism, and degenerative system.

In some embodiments, the VMAT2 inhibitor is administered to the patient to treat a disease or disorder chosen from:

ataxias or spinal muscular atrophies such as spinocerebellar ataxia type 17 (SCA17)/HDL4, ataxia, spinal muscular atrophy, amyotrophic lateral sclerosis, familial amyotrophic lateral sclerosis, bulbospinal muscular atrophy congenital, dentatorubral-pallidoluysian atrophy, hereditary motor neuron disease, and hereditary spastic paraplegia;

chorea such as benign hereditary chorea, chorea, chorea associated with mitochondrial disease/causes, chorea associated with Wilson's disease, chorea gravidarum, chorea-acanthocytosis, drug-induced chorea, hemiballism, rheumatic/Sydenham's chorea, and thyrotoxic chorea/hyperthyroid chorea;

congenital malformations, deformations or abnormalities such as Angelman syndrome, congenital neurological disorder, Aicardi's syndrome, neurofibromatosis, congenital facial nerve hypoplasia, Moebius II syndrome, Cockayne's syndrome, Sjogren-Larsson syndrome, Laurence-Moon-Bardet-Biedl syndrome, Fragile X syndrome, and Prader-Willi syndrome;

dementia such as AIDS-related dementia, Alzheimer's disease, congenital neurological degeneration, Lewy body dementia, micro-infarct dementia, pre-senile dementia, senile dementia, and vascular dementia;

diseases of oral cavity, salivary glands and jaws, such as glossodynia/burning mouth syndrome and temporomandibular joint disorder;

dyskinesia such as pharyngeal dyskinesia, dyskinesia, dyskinesia (neonatal), dyskinesia (oesophageal), levodopa-induced dyskinesia, paroxysmal kinesigenic dyskinesias, paroxysmal nonkinesigneic dyskinesias, and respiratory dyskinesia;

dystonia such as blepharospasm, buccoglossal syndrome, drug-induced acute dystonia, dystonia, early onset primary dystonia, genetic torsion dystonia, hand dystonia/writer's cramp, idiopathic nonfamilial dystonia, idiopathic orofacial dystonia/Meige's disease, laryngeal dystonia, oromandibular dystonia, and spasmodic torticollis/cervical dystonia;

endocrine, nutritional and metabolic diseases such as Wilson's Disease, diabetes mellitus, obesity, syndrome X, and Lesch-Nyhan syndromes;

epilepsy such as Baltic myoclonic epilepsy, benign familial neonatal convulsions, epilepsy, epilepsy congenital, Lafora's myoclonic epilepsy, severe myoclonic epilepsy of infancy, and convulsions;

habit and impulse disorders such as binge eating disorder, kleptomania, impulse control disorders, trichotillomania, intermittent explosive disorder, pathological gambling, and pyromania;

Huntingon's disease or related disorders such as Huntington's disease, Huntington's disease-like syndromes 1-3, Huntington's chorea, and X-linked McLeod Neurocanthocytosis syndrome;

mood or psychotic disorders such as schizophrenia, psychosis, mania, bipolar disorder, depression, and mood disorders;

other diseases or disorders such as fumbling, hypokinesia, hypokinesia (neonatal), movement disorder, rabbit syndrome, spasticity, up and down phenomenon, asthma, cancer, congenital nystagmus, familial hemiplegic migraine, fetal movement disorder, and rheumatoid arthritis;

neurotic, stress-related and somatoform disorders such as social anxiety disorder, panic disorder, generalized anxiety disorder, obsessive compulsive disorder, post-traumatic stress disorder, and psychogenic movement disorder;

other degenerative diseases of basal ganglia such as pantothethenate kinase-associated neurodegeneration, progressive supranuclear palsy, multiple system atrophy, dyslexia, basal ganglion degeneration, and neuroferritinopathy;

other extrapyramidal and movement disorders such as demiballismus, extrapyramidal disorder, essential tremor, geniospasm, hyperexplexia, akathisia, ballismus/hemiballism, myoclonus, and restless legs syndrome/Willis-Ekbom's syndrome;

other nervous system or motor function such as sleep-related bruxism, abnormal involuntary movement disorders, alien limb syndrome, Alzheimer's disease (agitation), clumsiness, clonic hemifacial spasm, olfactory nerve agenesis, congenital cranial nerve paralysis, exercise ataxia syndrome, familial periodic paralysis, congenital hemiparesis, fine motor delay, fine motor skill dysfunction, gross motor delay, multiple sclerosis, congenital flaccid paralysis, congenital Horner's syndrome, alternating hemiplegia of childhood, motor developmental delay, cerebral palsy, athetoid cerebral palsy, posturing, pseudoparalysis, psychomotor hyperactivity, bradykinesia, synkinesis, akinesia, Riley-Day syndrome, and athetosis;

Parkinson's/parkinsonism such as parkinsonism, drug-induced parkinsonism, micrographia, and Parkinson's disease;

demoralization including demoralization and subjective incompetence

pediatric-onset behavioral and emotional disorders such as attention deficit hyperactivity disorder, attention deficit disorder, hyperkinesia, hyperkinesia (neonatal), oppositional defiant disorder, provisional tic disorder, persistent (chronic) motor or vocal tic disorder, stereotypic movement disorder, stereotypy, and Tourette's syndrome;

pervasive developmental disorders such as autism spectrum disorders, Rett's syndrome, Asperger's syndrome, pervasive developmental disorder NOS, and dyslexia; and

substance abuse or dependence such as addiction disorders, alcoholism, cocaine dependence, illegal drug abuse, methamphetamine abuse, methamphetamine addiction/dependence, methamphetamine use disorder, morphine abuse, morphine-analogue abuse, nicotine dependence, polysubstance abuse, and prescription drug abuse.

In some embodiments, the patient has been determined to have 22q11.2 deletion syndrome. In some embodiments, the patient is predisposed to developing a psychiatric disorder due to the patient having 22q11.2 deletion syndrome. In some embodiments, the patient has been determined to have COMT haploinsufficiency. In some embodiments, the patient is predisposed to developing a psychiatric disorder due to the patient having COMT haploinsufficiency.

In some embodiments, the patient has been determined to have Velocardiofacial syndrome (VCFS). In some embodiments, the patient with Velocardiofacial syndrome has a 3 Mb deletion. In some embodiments, the 3 Mb deletion comprises the deletion of COMT and TBX1. In some embodiments, the patient with Velocardiofacial syndrome has a 1.5 Mb deletion. In some embodiments, the 1.5 Mb deletion comprises the deletion of TBX1 and COMT.

In some embodiments, the method or use further comprises monitoring the patient for one or more exposure-related adverse reactions. In some embodiments, the one or more exposure-related adverse reactions is chosen from hypersensitivity reactions. In some embodiments, the one or more exposure-related adverse reactions is chosen from hypersensitivity reactions with or without dermatological reactions. In some embodiments, the one or more exposure-related adverse reactions is chosen from hypersensitivity reactions with dermatological reactions. In some embodiments, the one or more exposure-related adverse reactions is chosen from hypersensitivity reactions without dermatological reactions. In some embodiments, the one or more exposure-related adverse reactions is chosen from allergic dermatitis, angioedema, pruritis, and urticaria.

In some embodiments, hypersensitivity is Type I hypersensitivity. In some embodiments, hypersensitivity is Type IV hypersensitivity.

In some embodiments, the one or more exposure-related adverse reactions is chosen from urticaria, pruritus, allergic dermatitis, and angioedema. In some embodiments, the one or more exposure-related adverse reactions is chosen from urticaria, allergic dermatitis, and angioedema. In some embodiments, the one or more exposure-related adverse reactions is hypersensitivity reaction and rash. In some embodiments, the one or more exposure-related adverse reactions is rash. In some embodiments, the one or more exposure-related adverse reactions is chosen from rash, urticaria, and reactions consistent with angioedema.

In some embodiments, the one or more exposure-related adverse reactions is chosen from reactions consistent with angioedema. In some embodiments, the one or more exposure-related adverse reactions that are consistent with angioedema are chosen from swelling of the face, lips, and mouth, and dyspnea.

In some embodiments, the patient in need thereof who is at increased risk of one or more exposure-related adverse reactions has a history of allergies. In some embodiments, the patient has a history of allergies to one or more drugs, e.g., penicillin or paroxetine; to one or more types of food, e.g., eggs, milk, peanuts, tree nuts, fish, shellfish, wheat or soy; and/or to cats. In some embodiments, the patient has a history of hives.

In some embodiments, the method or use further comprises administering to the patient that is experiencing one or more exposure-related adverse reactions one or more medications chosen from steroids and antihistamines. In some embodiments, the steroid is a systemic glucocorticoid, such as prednisone. In some embodiments, the steroid is a hydrocortisone cream. In some embodiments, the antihistamine is diphenhydramine.

In some embodiments, the patient is also being administered digoxin, and the method further comprises administering to the patient a therapeutically effective amount of the VMAT2 inhibitor. In some embodiments, the dose of digoxin is reduced.

In some embodiments, the patient is also being administered digoxin, and the method further comprises administering to the patient a therapeutically effective amount of the VMAT2 inhibitor, subsequently determining that the patient is to begin treatment with digoxin, and continuing administration of the therapeutically effective amount of the VMAT2 inhibitor to the patient. In some embodiments, the dose of digoxin is reduced.

In some embodiments, the patient is also being administered digoxin, and the method further comprises administering a therapeutically effective amount of the VMAT2 inhibitor to the patient, wherein the administration produces a mean digoxin Cₘₐₓ that is about 1.5 to 2.5 fold higher than the mean digoxin Cₘₐₓ for a patient who is administered digoxin alone and/or a mean digoxin AUC_{0-∞} that is about 1 to about 2 fold higher than the mean digoxin AUC_{0-∞} for a patient who is administered digoxin alone.

In some embodiments, the patient is a poor metabolizer of cytochrome P450 2D6 (CYP2D6). In certain embodiments, the patient has a CYP2D6 poor metabolizer genotype. In certain embodiments, the CYP2D6 poor metabolizer genotype is chosen from the CYP2D6G1846A genotype or the CYP2D6C100T genotype. In certain embodiments, the CYP2D6 poor metabolizer genotype is one of the CYP2D6G1846A (AA) genotype or the CYP2D6G1846A (AG) genotype. In certain embodiments, the CYP2D6 poor metabolizer genotype is the CYP2D6G1846A (AA) genotype. In certain embodiments, the CYP2D6 poor metabolizer genotype is one of the CYP2D6C100T (TT) genotype or the CYP2D6C100T (CT) genotype. In certain embodiments, the CYP2D6 poor metabolizer genotype is the CYP2D6C100T (TT) genotype. In some embodiments, the patient with CYP2D6 poor metabolizer genotype is administered an amount of about 40 mg of valbenazine free base once daily.

In some embodiments, administration to a patient who is a poor metabolizer of CYP2D6 results in increased exposure of (+)-α-3-isobutyl-9,10-dimethoxy-1,3,4,6,7,11b-hexahydro-2H-pyrido[2,1-a]isoquinolin-2-ol. In some embodiments, administration to a patient who is a poor metabolizer of CYP2D6 results in increased risk of one or more clinically significant parkinson-like signs or symptoms. In some embodiments, administration to a patient who is a poor metabolizer of CYP2D6 results in prolongation of the patient's QT interval. In certain embodiments, the therapeutically effective amount is less than the amount that is administered to a patient who is not a CYP2D6 poor metabolizer. In certain embodiments, the therapeutically effective amount is the same amount as that administered to a patient who is not a CYP2D6 poor metabolizer.

Also provided herein is a pharmaceutical composition comprising the VMAT2 inhibitor as an active pharmaceutical ingredient, in combination with one or more pharmaceutically acceptable carriers or excipients.

Also provided herein is a composition comprising a therapeutically effective amount of the VMAT2 inhibitor chosen from valbenazine, (+)-α-3-isobutyl-9,10-dimethoxy-1,3,4,6,7,11b-hexahydro-2H-pyrido[2,1-a]isoquinolin-2-ol, and a pharmaceutically acceptable salt and/or isotopic variant thereof for treating a patient with a disease or disorder, wherein the patient is being monitored for one or more clinically significant parkinson-like signs or symptoms.

In some embodiments, the patient is an adult.

In some embodiments, the composition further comprises the step of discontinuing administration of the composition based on the patient's ability to tolerate one or more clinically significant parkinson-like signs or symptoms after a reduced amount or dose of the VMAT2 inhibitor is administered. In some embodiments, the administration is discontinued for a first period of time, such as at least one week, e.g., one, two, three or four weeks, and then administration is continued with a composition at a reduced dose of the VMAT2 inhibitor.

In some embodiments, the composition further comprises the step of informing the patient or a medical care worker that administration of the VMAT2 inhibitor to the patient may result in one or more clinically significant parkinson-like signs or symptoms. In some embodiments, the composition further comprises the step of informing the patient or a medical care worker that administration of the VMAT2 inhibitor to the patient may result in increased risk of the one or more clinically significant parkinson-like signs or symptoms. In some embodiments, the composition further comprises the step of informing the patient or a medical care worker that administration of the VMAT2 inhibitor to the patient may result in worsening of pre-existing parkinsonism or parkinson-like signs or symptoms. In some embodiments, the composition further comprises the step of informing the patient to report to a medical care worker any clinically significant parkinson-like signs or symptoms.

In some embodiments, the composition is not administered to the patient having pre-existing parkinsonism. In some embodiments, the composition further comprises the step of determining whether the patient has pre-existing parkinsonism prior to initiation of treatment with the VMAT2 inhibitor.

In some embodiments, the composition comprises the step of administering to the patient that is experiencing one or more clinically significant parkinson-like signs or symptoms one or more medications used to treat Parkinson disease. In some embodiments, the medication is a dopamine decarboxylase inhibitor in combination with a dopamine precursor, e.g., the dopamine decarboxylase inhibitor carbidopa or benserazide in combination with the dopamine precursor levodopa. In some embodiments, the medication is a catechol-o-methyltransferase (COMT) inhibitor, such as entacapone, tolcapone, or opicapone. In some embodiments, the medication is a dopamine decarboxylase inhibitor in combination with a dopamine precursor in further combination with a COMT inhibitor. In some embodiments, the medication is a dopamine agonist, such as pramipexole, ropinirole, apomorphine, bromocriptine, or rotigotine. In some embodiments, the medication is a monoamine oxidase B inhibitor such as selegiline, rasagiline, or safinamide. In some embodiments, the medication is amantadine. In some embodiments, the medication is an anticholinergic drug, such as trihexyphenidyl or benztropine. In some embodiments, the medication is chosen from levodopa, carbidopa, and opicapone. In some embodiments, the medication is chosen from levodopa, carbidopa, and entacapone. In some embodiments, the medication is levodopa in combination with carbidopa and opicapone. In some embodiments, the medication is chosen from rivastigmine, cariprazine, and paliperidone.

In some embodiments, prior to administration of the therapeutically effective amount of the VMAT2 inhibitor, the patient had a Simpson-Angus Scale score of <0.3. In some embodiments, after administration of the therapeutically effective amount of the VMAT2 inhibitor, the patient had a change in Simpson-Angus Scale score of >1.

In some embodiments, prior to the administration, the patient is at increased risk of experiencing one or more clinically significant parkinson-like signs or symptoms.

In some embodiments, the patient at increased risk of experiencing clinically significant parkinson-like signs or symptoms is a patient who is being co-administered one or more antipsychotics, antidepressants, antiepileptics, or other drugs that are known to possibly cause parkinsonism. In some embodiments, the patient is being co-administered one or more drugs chosen from amlodipine, atropine, benztropine, clonazepam, clozapine, fluoxetine, gabapentin, Lamictal, lisinopril, lithium, lurasidone, olanzapine, oxycodone, paliperidone, pregabalin, prazosin, quetiapine, tiotixene, tizanidine, valproic acid, and valproate. In some embodiments, the patient being co-administered one or more other drugs is clinically stable.

In some embodiments, the patient at increased risk of experiencing clinically significant parkinson-like signs or symptoms is a patient who is being co-administered one or more antipsychotics. In some embodiments, the antipsychotic drug is a typical antipsychotic drug. In some embodiments, the typical antipsychotic drug is fluphenazine, haloperidol, loxapine, molindone, perphenazine, pimozide, sulpiride, thioridazine, or trifluoperazine. In some embodiments, the antipsychotic drug is an atypical antipsychotic drug. In some embodiments, the atypical antipsychotic drug is aripiprazole, asenapine, clozapine, iloperidone, olanzapine, paliperidone, quetiapine, risperidone, or ziprasidone. In some embodiments, the atypical antipsychotic drug is clozapine.

In some embodiments, the patient at increased risk of experiencing clinically significant parkinson-like signs or symptoms is a patient having pre-existing parkinsonism. In some embodiments, the patient at increased risk of experiencing clinically significant parkinson-like signs or symptoms is a patient having pre-existing Parkinson's disease (also referred to as idiopathic Parkinson's disease.) In some embodiments, the patient at increased risk of experiencing clinically significant parkinson-like signs or symptoms is a patient having a pre-existing condition chosen from corticobasal degeneration, dementia with Lewy Bodies, drug-induced parkinsonism, essential tremor, multiple system atrophy, progressive supranuclear palsy, and vascular parkinsonism. In some embodiments, the patient at increased risk of experiencing clinically significant parkinson-like signs or symptoms is a patient having a pre-existing gait disorders. In some embodiments, the patient having a pre-existing condition is clinically stable.

In some embodiments, prior to administration of the therapeutically effective amount of the VMAT2 inhibitor, the patient had a Simpson-Angus Scale score of ≥0.3. In some embodiments, after administration of the therapeutically effective amount of the VMAT2 inhibitor, the patient had a change in Simpson-Angus Scale score of >1.

In some embodiments, the one or more clinically significant parkinson-like signs or symptoms is chosen from difficulty moving or loss of ability to move muscles voluntarily, tremor, gait disturbances and drooling. In some embodiments, the one or more clinically significant parkinson-like signs or symptoms is chosen from akinesia, severe tremor, gait disturbances (shuffling, festination) and drooling. In some embodiments, the one or more clinically significant parkinson-like signs or symptoms is chosen from falls, gait disturbances, tremor, drooling and hypokinesia. In some embodiments, the one or more clinically significant parkinson-like signs or symptoms is chosen from shaking, body stiffness, trouble moving or walking and trouble keeping balance.

In some embodiments, the one or more clinically significant parkinson-like signs or symptoms occurs within the first two weeks after starting or increasing the dose of the VMAT2 inhibitor administered to the patient.

In some embodiments, the composition further comprises the step of selecting a patient for continued treatment with the VMAT2 inhibitor if the patient has not experienced one or more clinically significant parkinson-like signs or symptoms within the first two weeks after starting or increasing the dose of the VMAT2 inhibitor administered to the patient.

In some embodiments, the one or more clinically significant parkinson-like signs or symptoms occurs within the first two weeks of administration of the VMAT2 inhibitor. In some embodiments, the one or more clinically significant parkinson-like signs or symptoms occurs within the first two weeks of increasing the amount of the VMAT2 inhibitor administered to the patient.

In some embodiments, the severity of at least one of the one or more clinically significant parkinson-like signs or symptoms is reduced after discontinuing administration of the VMAT2 inhibitor. In some embodiments, at least one of the one or more clinically significant parkinson-like signs or symptoms is resolved after discontinuing administration of the VMAT2 inhibitor. In a further embodiment, the VMAT2 inhibitor is valbenazine ditosylate.

In some embodiments, the VMAT2 inhibitor is chosen from valbenazine and pharmaceutically acceptable salts and/or isotopic variants thereof. In some embodiments, the VMAT2 inhibitor is valbenazine, or a pharmaceutically acceptable salt thereof. In some embodiments, the VMAT2 inhibitor is a valbenazine salt. In some embodiments, the VMAT2 inhibitor is a valbenazine tosylate salt. In some embodiments, the VMAT2 inhibitor is a ditosylate salt of valbenazine. In some embodiments, the ditosylate salt of valbenazine is amorphous.

In some embodiments, the ditosylate salt of valbenazine is substantially amorphous. In some embodiments, the ditosylate salt of valbenazine is substantially crystalline. In some embodiments, the crystalline ditosylate salt of valbenazine has an XRPD diffraction pattern comprising X-ray diffraction peaks at two-theta angles of 6.3, 17.9, and 19.7°±0.2°. In some embodiments, the crystalline ditosylate salt of valbenazine has an XRPD diffraction pattern comprising X-ray diffraction peaks at two-theta angles of 6.3 and 17.9°±0.2°. In some embodiments, the crystalline ditosylate salt of valbenazine has an XRPD diffraction pattern comprising X-ray diffraction peaks at two-theta angle of 6.3°±0.2°. In some embodiments, crystalline Form I has a DSC thermogram comprising an endothermic event with an onset temperature of about 240° C. and a peak at about 243° C.

In some embodiments, the composition is administered orally. In some embodiments, the composition is administered in the form of a tablet or capsule.

In some embodiments, the composition is administered with or without food.

In some embodiments, the VMAT2 inhibitor is administered in an amount equivalent to between about 20 mg and about 160 mg, between about 20 mg and about 150 mg, between 20 mg and about 140 mg, between about 20 mg and about of 130 mg, between about 20 mg and 120 mg, between about 30 mg and 120 mg, or between about 40 mg and about 120 mg once daily. In some embodiments, the VMAT2 inhibitor is administered in an amount equivalent to between about 20 mg and about 100 mg once daily. In some embodiments, the VMAT2 inhibitor is administered in an amount equivalent to about 20 mg once daily. In some embodiments, the VMAT2 inhibitor is administered in an amount equivalent to about 40 mg once daily. In some embodiments, the VMAT2 inhibitor is administered in an amount equivalent to about 50 mg once daily. In some embodiments, the VMAT2 inhibitor is administered in an amount equivalent to about 60 mg once daily. In some embodiments, the VMAT2 inhibitor is administered in an amount equivalent to about 70 mg once daily. In some embodiments, the VMAT2 inhibitor is administered in an amount equivalent to about 80 mg once daily. In some embodiments, the VMAT2 inhibitor is administered in an amount equivalent to about 100 mg once daily. In some embodiments, the VMAT2 inhibitor is administered in an amount equivalent to about 120 mg once daily.

In some embodiments, the VMAT2 inhibitor is administered in an amount equivalent to between about 20 mg and about 160 mg, between about 20 mg and about 150 mg, between 20 mg and about 140 mg, between about 20 mg and about of 130 mg, between about 20 mg and 120 mg, between about 30 mg and 120 mg, or between about 40 mg and about 120 mg. In some embodiments, the VMAT2 inhibitor is administered in an amount equivalent to between about 20 mg and about 100 mg. In some embodiments, the VMAT2 inhibitor is administered in an amount equivalent to about 20 mg. In some embodiments, the VMAT2 inhibitor is administered in an amount equivalent to about 40 mg. In some embodiments, the VMAT2 inhibitor is administered in an amount equivalent to about 50 mg. In some embodiments, the VMAT2 inhibitor is administered in an amount equivalent to about 60 mg. In some embodiments, the VMAT2 inhibitor is administered in an amount equivalent to about 70 mg. In some embodiments, the VMAT2 inhibitor is administered in an amount equivalent to about 80 mg. In some embodiments, the VMAT2 inhibitor is administered in an amount equivalent to about 100 mg. In some embodiments, the VMAT2 inhibitor is administered in an amount equivalent to about 120 mg.

In some embodiments, the therapeutically effective amount is an amount equivalent to between about 20 mg and about 160 mg, between about 20 mg and about 150 mg, between 20 mg and about 140 mg, between about 20 mg and about of 130 mg, between about 20 mg and 120 mg, between about 30 mg and 120 mg, or between about 40 mg and about 120 mg valbenazine free base once daily. In some embodiments, the therapeutically effective amount is an amount equivalent to between about 20 mg and about 100 mg of valbenazine free base once daily. In some embodiments, the therapeutically effective amount is an amount equivalent to about 20 mg of valbenazine free base once daily. In some embodiments, the therapeutically effective amount is an amount equivalent to about 40 mg of valbenazine free base once daily. In some embodiments, the therapeutically effective amount is an amount equivalent to about 50 mg of valbenazine free base once daily. In some embodiments, the therapeutically effective amount is an amount equivalent to about 60 mg of valbenazine free base once daily. In some embodiments, the therapeutically effective amount is an amount equivalent to about 70 mg of valbenazine free base once daily. In some embodiments, the therapeutically effective amount is an amount equivalent to about 80 mg of valbenazine free base once daily. In some embodiments, the therapeutically effective amount is an amount equivalent to about 100 mg of valbenazine free base once daily. In some embodiments, the therapeutically effective amount is an amount equivalent to about 120 mg of valbenazine free base once daily.

In some embodiments, the therapeutically effective amount is an amount equivalent to between about 20 mg and about 160 mg, between about 20 mg and about 150 mg, between 20 mg and about 140 mg, between about 20 mg and about of 130 mg, between about 20 mg and 120 mg, between about 30 mg and 120 mg, or between about 40 mg and about 120 mg of valbenazine free base. In some embodiments, the therapeutically effective amount is an amount equivalent to between about 20 mg and about 100 mg of valbenazine free base. In some embodiments, the therapeutically effective amount an amount equivalent to about 20 mg of valbenazine free base. In some embodiments, the therapeutically effective amount is an amount equivalent to about 40 mg of valbenazine free base. In some embodiments, the therapeutically effective amount is an amount equivalent to about 50 mg of valbenazine free base. In some embodiments, the therapeutically effective amount is an amount equivalent to about 60 mg of valbenazine free base. In some embodiments, the therapeutically effective amount is an amount equivalent to about 70 mg of valbenazine free base. In some embodiments, the therapeutically effective amount is an amount equivalent to about 80 mg of valbenazine free base. In some embodiments, the therapeutically effective amount is an amount equivalent to about 100 mg of valbenazine free base. In some embodiments, the therapeutically effective amount is an amount equivalent to about 120 mg of valbenazine free base.

In some embodiments, the reduced amount of the VMAT2 inhibitor is reduced by at least about 10% of a therapeutically effective amount that would be administered to a patient who does not experience one or more clinically significant parkinson-like signs or symptoms as a result of administration of the VMAT2 inhibitor. In some embodiments, the reduced amount of the VMAT2 inhibitor is reduced by at least about 20% of the therapeutically effective amount. In some embodiments, the reduced amount of the VMAT2 inhibitor is reduced by at least about 30% of the therapeutically effective amount. In some embodiments, the reduced amount of the VMAT2 inhibitor is reduced by at least about 40% of the therapeutically effective amount. In some embodiments, the reduced amount of the VMAT2 inhibitor is reduced by at least about 50% of the therapeutically effective amount. In some embodiments, the reduced amount of the VMAT2 inhibitor is reduced by at least about 60% of the therapeutically effective amount. In some embodiments, the reduced amount of the VMAT2 inhibitor is reduced by at least about 70% of the therapeutically effective amount. In some embodiments, the reduced amount of the VMAT2 inhibitor is reduced by at least about 80% of the therapeutically effective amount. In some embodiments, the reduced amount of the VMAT2 inhibitor is reduced by at least about 90% of the therapeutically effective amount.

In certain embodiments, the reduced amount of the VMAT2 inhibitor is 10-90% less than the amount that would be administered to a patient who does not experience one or more clinically significant parkinson-like signs or symptoms as a result of administration of the VMAT2 inhibitor. In certain embodiments, the reduced amount of the VMAT2 inhibitor is 20-80% less than the amount that would be administered to a patient who does not experience one or more clinically significant parkinson-like signs or symptoms as a result of administration of the VMAT2 inhibitor. In certain embodiments, the reduced amount of the VMAT2 inhibitor is 30-70% less than the amount that would be administered to a patient who does not experience one or more clinically significant parkinson-like signs or symptoms as a result of administration of the VMAT2 inhibitor. In certain embodiments, the reduced amount of the VMAT2 inhibitor is 40-60% less than the amount that would be administered to a patient who does not experience one or more clinically significant parkinson-like signs or symptoms as a result of administration of the VMAT2 inhibitor. In certain embodiments, the reduced amount of the VMAT2 inhibitor is about 50% less than the amount that would be administered to a patient who does not experience one or more clinically significant parkinson-like signs or symptoms as a result of administration of the VMAT2 inhibitor. In some embodiments, the amount that would be administered to a patient who does not experience one or more clinically significant parkinson-like signs or symptoms as a result of administration of the VMAT2 inhibitor is between about 20 mg to about 120 mg per day. In some embodiments, the amount that would be administered to a patient who does not experience one or more clinically significant parkinson-like signs or symptoms as a result of administration of the VMAT2 inhibitor is about 40 mg, about 60 mg, or 80 mg once daily.

In certain embodiments, the reduced amount of the VMAT2 inhibitor is between about 10 mg to about 80 mg, between about 10 mg to about 70 mg, between about 10 mg to about 60 mg, between about 10 mg to about 50 mg, between about 10 mg to about 40 mg, between about 10 mg to about 30 mg, between about 20 mg to about 80 mg, between about 20 mg to about 70 mg, between about 20 mg to about 60 mg, between about 20 mg to about 50 mg, between about 20 mg to about 40 mg, between about 20 mg to about 30 mg, between about 30 mg to about 80 mg, between about 30 mg to about 70 mg, between about 30 mg to about 60 mg, between about 30 mg to about 50 mg, between about 30 mg to about 40 mg of valbenazine per day. In a further embodiment, the VMAT 2 inhibitor is administered once daily.

In certain embodiments, the reduced amount or dose of the VMAT2 inhibitor is about 10 mg, about 15 mg, about 20 mg, about 25 mg, about 30 mg, about 35 mg, about 40 mg, about 45 mg, about 50 mg, about 55 mg, about 60 mg of valbenazine per day. In a further embodiment, the VMAT 2 inhibitor is administered once daily.

For example, wherein the amount that would be administered to a patient who does not experience one or more clinically significant parkinson-like signs or symptoms as a result of administration of the VMAT2 inhibitor is about 40 mg per day, an individual may receive a reduced dosage of about 36, 35, 32, 30, 28, 25, 24, 20, 16, 12, 8, or 4 mg per day. Likewise, wherein the amount that would be administered to a patient who does not experience one or more clinically significant parkinson-like signs or symptoms as a result of administration of the VMAT2 inhibitor is about 60 mg per day, an individual may receive a reduced dosage of about 56, 50, 48, 45, 40, 32, 30, 24, 16, or 8 per day. Likewise, wherein the amount that would be administered to a patient who does not experience one or more clinically significant parkinson-like signs or symptoms as a result of administration of the VMAT2 inhibitor is about 80 mg per day, an individual may receive a reduced dosage of about 72, 64, 60, 56, 50, 48, 45, 40, 32, 30, 24, 20, 16, or 8 per day. In a further embodiment, the VMAT2 inhibitor is administered once daily.

For example, wherein the dosage administered to a patient who does not experience one or more clinically significant parkinson-like signs or symptoms as a result of administration of the VMAT2 inhibitor is about 40 mg per day, an individual may receive a reduced dosage of about 4-36 mg per day, about 8-32 mg per day, about 12-28 mg per day, about 16-24 mg per day, or in certain embodiments, about 20 mg per day. In some embodiments, wherein the dosage administered to a patient who does not experience one or more clinically significant parkinson-like signs or symptoms as a result of administration of the VMAT2 inhibitor is about 80 mg per day, an individual may receive a reduced dosage of about 8-72 mg per day, about 16-64 mg per day, about 24-56 mg per day, about 32-48 mg per day, or in certain embodiments, about 24 mg per day. In a further embodiment, the VMAT2 inhibitor is administered once daily.

In some embodiments, wherein the dosage administered to a patient who does not experience one or more clinically significant parkinson-like signs or symptoms as a result of administration of the VMAT2 inhibitor is about 40 mg per day, an individual may receive a reduced dosage of about 5-35 mg per day, about 10-30 mg per day, about 15-30 mg per day, about 15-25 mg per day, or in certain embodiments, about 20 mg per day or about 30 mg per day. In some embodiments, wherein the dosage administered to a patient who does not experience one or more clinically significant parkinson-like signs or symptoms as a result of administration of the VMAT2 inhibitor is about 40 mg per day, an individual may receive the same dosage of about 40 mg every other day, a reduced dosage of about 5-35 mg every other day, about 10-30 mg every other day, about 15-30 mg every other day, about 15-25 mg every other day, or in certain embodiments, about 20 mg every other day or about 30 mg every other day.

In some embodiments, wherein the dosage administered to a patient who does not experience one or more clinically significant parkinson-like signs or symptoms as a result of administration of the VMAT2 inhibitor is about 60 mg per day, an individual may receive a reduced dosage of about 5-75 mg per day, about 10-70 mg per day, about 15-65 mg per day, about 20-60 mg per day, about 25-55 mg per day, about 30-50 mg per day, or in certain embodiments, about 40 mg per day. In some embodiments, wherein the dosage administered to a patient who does not experience one or more clinically significant parkinson-like signs or symptoms as a result of administration of the VMAT2 inhibitor is about 60 mg per day, an individual may receive the same dosage of about 60 mg every other day, a reduced dosage of about 5-75 mg every other day, about 10-70 mg every other day, about 15-65 mg every other day, about 20-60 mg every other day, about 25-55 mg every other day, about 30-50 mg every other day, or in certain embodiments, about 40 mg every other day.

In some embodiments, wherein the dosage administered to a patient who does not experience one or more clinically significant parkinson-like signs or symptoms as a result of administration of the VMAT2 inhibitor is about 80 mg per day, an individual may receive a reduced dosage of about 5-75 mg per day, about 10-70 mg per day, about 15-65 mg per day, about 20-60 mg per day, about 25-55 mg per day, about 30-50 mg per day, or in certain embodiments, about 40 mg per day. In some embodiments, wherein the dosage administered to a patient who does not experience one or more clinically significant parkinson-like signs or symptoms as a result of administration of the VMAT2 inhibitor is about 80 mg per day, an individual may receive the same dosage of about 80 mg every other day, a reduced dosage of about 5-75 mg every other day, about 10-70 mg every other day, about 15-65 mg every other day, about 20-60 mg every other day, about 25-55 mg every other day, about 30-50 mg every other day, or in certain embodiments, about 40 mg every other day.

In some embodiments, wherein the dosage administered to a patient who does not experience one or more clinically significant parkinson-like signs or symptoms as a result of administration of the VMAT2 inhibitor is about 80 mg per day, an individual may receive a reduced dosage of about 10-75 mg per day, about 20-70 mg per day, about 30-65 mg per day, about 40-65 mg per day, about 45-45 mg per day, or in certain embodiments, about 60 mg per day. In some embodiments, wherein the dosage administered to a patient who does not experience one or more clinically significant parkinson-like signs or symptoms as a result of administration of the VMAT2 inhibitor is about 80 mg per day, an individual may receive the same dosage of about 80 mg every other day, a reduced dosage of about 10-75 mg every other day, about 20-70 mg every other day, about 30-65 mg every other day, about 40-65 mg every other day, about 45-45 mg every other day, or in certain embodiments, about 60 mg every other day.

In some embodiments, wherein a patient who does not experience one or more clinically significant parkinson-like signs or symptoms as a result of administration of a first therapeutically effective amount of a VMAT2 inhibitor, the patient may receive a second therapeutically effective amount, wherein the second therapeutically effective amount may be the same or a higher dose compared to the first therapeutically effective amount. In some further embodiments, the first therapeutically effective amount is an amount between about 30 and 100 mg and the second therapeutically effective amount is an amount between about 40 mg and about 120 mg. In some embodiments, the first therapeutically effective amount is about 30 mg, about 40 mg, about 50 mg, about 60 mg, about 70 mg, about 80 mg, about 90 mg and about 100 mg of valbenazine free base per day. In some embodiments, the second therapeutically effective amount is about 30 mg, about 40 mg, about 50 mg, about 60 mg, about 70 mg, about 80 mg, about 90 mg, about 100 mg, about 110 mg, and about 120 mg of valbenazine free base per day. In some embodiments, the first therapeutically effective amount is about 40 mg of valbenazine free base per day, and the second therapeutically effective amount is about 40 mg of valbenazine free base per day. In some embodiments, the first therapeutically effective amount is about 40 mg of valbenazine free base per day, and the second therapeutically effective amount is about 60 mg of valbenazine free base per day. In some embodiments, the first therapeutically effective amount is about 40 mg of valbenazine free base per day, and the second therapeutically effective amount is about 80 mg of valbenazine free base per day. In some embodiments, the first therapeutically effective amount is about 60 mg of valbenazine free base per day, and the second therapeutically effective amount is about 60 mg of valbenazine free base per day. In some embodiments, the first therapeutically effective amount is about 60 mg of valbenazine free base per day, and the second therapeutically effective amount is about 80 mg of valbenazine free base per day. In some embodiments, the first therapeutically effective amount is about 80 mg of valbenazine free base per day, and the second therapeutically effective amount is about 80 mg of valbenazine free base per day.

In some embodiments, wherein a patient who experiences one or more clinically significant parkinson-like signs or symptoms as a result of administration of a first therapeutically effective amount of a VMAT2 inhibitor, the patient may receive a second therapeutically effective amount, wherein the second therapeutically effective amount is less than the amount in the first therapeutically effective amount. In some further embodiments, the first therapeutically effective amount is an amount between about 30 and 100 mg and the second therapeutically effective amount is an amount between about 40 mg and about 120 mg. In some embodiments, the first therapeutically effective amount is about 30 mg, about 40 mg, about 50 mg, about 60 mg, about 70 mg, about 80 mg, about 90 mg and about 100 mg of valbenazine free base per day. In some embodiments, the second therapeutically effective amount is about 10 mg, about 20 mg, about 30 mg, about 40 mg, about 50 mg, or about 60 mg of valbenazine free base per day. In some embodiments, the first therapeutically effective amount is about 40 mg of valbenazine free base per day, and the second therapeutically effective amount is about 30 mg of valbenazine free base per day. In some embodiments, the first therapeutically effective amount is about 60 mg of valbenazine free base per day, and the second therapeutically effective amount is about 40 mg of valbenazine free base per day. In some embodiments, the first therapeutically effective amount is about 80 mg of valbenazine free base per day, and the second therapeutically effective amount is about 40 mg of valbenazine free base per day. In some embodiments, the first therapeutically effective amount is about 80 mg of valbenazine free base per day, and the second therapeutically effective amount is about 60 mg of valbenazine free base per day. In some embodiments, the first therapeutically effective amount is about 60 mg of valbenazine free base per day, and the second therapeutically effective amount is about 30 mg of valbenazine free base per day. In some embodiments, the first therapeutically effective amount is about 60 mg of valbenazine free base per day, and the second therapeutically effective amount is about 20 mg of valbenazine free base per day.

In some embodiments, the composition is administered in an amount sufficient to achieve a maximal blood plasma concentration (Cmax) of (+)-α-DHTBZ of between about 15 ng to about 60 ng per mL plasma and a minimal blood plasma concentration (Cmin) of approximately between about at least 33%-50% of the Cmax over a 12 hour period. In some embodiments, the VMAT2 inhibitor is administered in an amount sufficient to achieve: (i) a therapeutic concentration range of about 15 ng to about 60 ng of (+)-α-DHTBZ per mL plasma; and (ii) a threshold concentration of at least 15 ng (+)-α-DHTBZ per mL plasma over a period of about 8 hours to about 24 hours.

In some embodiments, the composition is administered for a first period of time in a first amount and then the amount is increased to a second amount. In some embodiments, the first period of time is a week. In some embodiments, the first period of time is more than one week, such as two weeks, three weeks, or four weeks. In some embodiments, the first period of time is one month, two months, three months or more. In some embodiments, the first amount is equivalent to about 40 mg of valbenazine free base once daily. In some embodiments, the first amount is equivalent to about 60 mg of valbenazine free base once daily. In some embodiments, the second amount is equivalent to about 60 mg of valbenazine free base once daily. In some embodiments, the second amount is equivalent to about 80 mg of valbenazine free base once daily.

In some embodiments, the composition is administered for a first period of time in a first amount and then the amount is decreased to a second amount. In some embodiments, the first period of time is a week. In some embodiments, the first period of time is more than one week, such as two weeks, three weeks, or four weeks. In some embodiments, the first period of time is one month, two months, three months or more. In some embodiments, the first amount is equivalent to about 60 mg of valbenazine free base once daily. In some embodiments, the first amount is equivalent to about 80 mg of valbenazine free base once daily. In some embodiments, the second amount is equivalent to about 40 mg of valbenazine free base once daily. In some embodiments, the second amount is equivalent to about 60 mg of valbenazine free base once daily. In some embodiments, the first amount is equivalent to about 60 mg of valbenazine free base once daily and the second amount is equivalent to about 40 mg of valbenazine free base once daily. In some embodiments, the first amount is equivalent to about 80 g of valbenazine free base once daily and the second amount is equivalent to about 40 mg of valbenazine free base once daily. In some embodiments, the first amount is equivalent to about 80 mg of valbenazine free base once daily and the second amount is equivalent to about 60 mg of valbenazine free base once daily.

In some embodiments, the composition is administered for a first period of time in a first amount and the administration of the VMAT inhibitor is discontinued. In some embodiments, the first period of time is a week. In some embodiments, the first period of time is more than one week, such as two weeks, three weeks, or four weeks. In some embodiments, the first period of time is one month, two months, three months or more. In some embodiments, the first amount is equivalent to about 40 mg of valbenazine free base once daily. In some embodiments, the first amount is equivalent to about 60 mg of valbenazine free base once daily. In some embodiments, the first amount is equivalent to about 80 mg of valbenazine free base once daily.

In some embodiments, the composition is administered to the patient to treat a neurological or psychiatric disease or disorder. In some embodiments, the neurological or psychiatric disease or disorder is a hyperkinetic movement disorder, mood disorder, bipolar disorder, schizophrenia, schizoaffective disorder, mania in mood disorder, depression in mood disorder, treatment-refractory obsessive compulsive disorder, neurological dysfunction associated with Lesch-Nyhan syndrome, agitation associated with Alzheimer's disease, Fragile X syndrome or Fragile X-associated tremor-ataxia syndrome, autism spectrum disorder, Rett syndrome, or chorea-acanthocytosis.

In some embodiments, the neurological or psychiatric disease or disorder is a hyperkinetic movement disorder. In some embodiments, the hyperkinetic movement disorder is tardive dyskinesia. In some embodiments, the hyperkinetic movement disorder is a tic disorder. In some embodiments, the tic disorder is Tourette's Syndrome. In some embodiments, the hyperkinetic movement disorder is Huntington's disease. In some embodiments, the hyperkinetic movement disorder is choreiform movements, general dystonia, focal dystonia, and myoclonus movements. In some embodiments, the hyperkinetic movement disorder is chorea associated with Huntington's disease. In some embodiments, the hyperkinetic movement disorder is ataxia, chorea, dystonia, Huntington's disease, myoclonus, restless leg syndrome, or tremors. In some embodiments, the hyperkinetic movement disorder is a disease or disorder other than Huntington's disease. In some embodiments, the hyperkinetic movement disorder is a disease or disorder other than Huntington's disease and the VMAT2 inhibitor is deutetrabenazine or tetrabenazine.

In some embodiments, the neurological or psychiatric disease or disorder is a hyperkinetic movement disorder in patients with intellectual and developmental disability (IDD). In some embodiments, the hyperkinetic movement disorder is tardive dyskinesia in patients with intellectual and developmental disability (IDD). In some embodiments, the hyperkinetic movement disorder is a tic disorder in patients with intellectual and developmental disability (IDD). In some embodiments, the tic disorder is Tourette's Syndrome in patients with intellectual and developmental disability (IDD). In some embodiments, the hyperkinetic movement disorder is Huntington's disease in patients with intellectual and developmental disability (IDD). In some embodiments, the hyperkinetic movement disorder is choreiform movements, general dystonia, focal dystonia, and myoclonus movements in patients with intellectual and developmental disability (IDD). In some embodiments, the hyperkinetic movement disorder is chorea associated with Huntington's disease in patients with intellectual and developmental disability (IDD). In some embodiments, the hyperkinetic movement disorder is ataxia, chorea, dystonia, Huntington's disease, myoclonus, restless leg syndrome, or tremors in patients with intellectual and developmental disability (IDD). In some embodiments, the hyperkinetic movement disorder is a disease or disorder other than Huntington's disease in patients with intellectual and developmental disability (IDD). In some embodiments, the hyperkinetic movement disorder is a disease or disorder other than Huntington's disease and the VMAT2 inhibitor is deutetrabenazine or tetrabenazine in patients with intellectual and developmental disability (IDD).

In some embodiments, the intellectual and developmental disability (IDD) comprises intellectual disability and developmental disability. In some embodiments, the intellectual and developmental disability (IDD) is intellectual disability. In some embodiments, the intellectual and developmental disability (IDD) is developmental disability. In some embodiments, the intellectual and developmental disability (IDD) is characterized by the body parts or systems being affected. In a further embodiment, the body parts or systems is selected from nervous system, sensory system, metabolism, and degenerative system.

In some embodiments, the composition is administered to the patient to treat a disease or disorder chosen from:

ataxias or spinal muscular atrophies such as spinocerebellar ataxia type 17 (SCA17)/HDL4, ataxia, spinal muscular atrophy, amyotrophic lateral sclerosis, familial amyotrophic lateral sclerosis, bulbospinal muscular atrophy congenital, dentatorubral-pallidoluysian atrophy, hereditary motor neuron disease, and hereditary spastic paraplegia;

chorea such as benign hereditary chorea, chorea, chorea associated with mitochondrial disease/causes, chorea associated with Wilson's disease, chorea gravidarum, chorea-acanthocytosis, drug-induced chorea, hemiballism, rheumatic/Sydenham's chorea, and thyrotoxic chorea/hyperthyroid chorea;

congenital malformations, deformations or abnormalities such as Angelman syndrome, congenital neurological disorder, Aicardi's syndrome, neurofibromatosis, congenital facial nerve hypoplasia, Moebius II syndrome, Cockayne's syndrome, Sjogren-Larsson syndrome, Laurence-Moon-Bardet-Biedl syndrome, Fragile X syndrome, and Prader-Willi syndrome;

dementia such as AIDS-related dementia, Alzheimer's disease, congenital neurological degeneration, Lewy body dementia, micro-infarct dementia, pre-senile dementia, senile dementia, and vascular dementia;

diseases of oral cavity, salivary glands and jaws, such as glossodynia/burning mouth syndrome and temporomandibular joint disorder;

dyskinesia such as pharyngeal dyskinesia, dyskinesia, dyskinesia (neonatal), dyskinesia (oesophageal), levodopa-induced dyskinesia, paroxysmal kinesigenic dyskinesias, paroxysmal nonkinesigneic dyskinesias, and respiratory dyskinesia;

dystonia such as blepharospasm, buccoglossal syndrome, drug-induced acute dystonia, dystonia, early onset primary dystonia, genetic torsion dystonia, hand dystonia/writer's cramp, idiopathic nonfamilial dystonia, idiopathic orofacial dystonia/Meige's disease, laryngeal dystonia, oromandibular dystonia, and spasmodic torticollis/cervical dystonia;

endocrine, nutritional and metabolic diseases such as Wilson's Disease, diabetes mellitus, obesity, syndrome X, and Lesch-Nyhan syndromes;

epilepsy such as Baltic myoclonic epilepsy, benign familial neonatal convulsions, epilepsy, epilepsy congenital, Lafora's myoclonic epilepsy, severe myoclonic epilepsy of infancy, and convulsions;

habit and impulse disorders such as binge eating disorder, kleptomania, impulse control disorders, trichotillomania, intermittent explosive disorder, pathological gambling, and pyromania;

Huntingon's disease or related disorders such as Huntington's disease, Huntington's disease-like syndromes 1-3, Huntington's chorea, and X-linked McLeod Neurocanthocytosis syndrome;

mood or psychotic disorders such as schizophrenia, psychosis, mania, bipolar disorder, depression, and mood disorders;

other diseases or disorders such as fumbling, hypokinesia, hypokinesia (neonatal), movement disorder, rabbit syndrome, spasticity, up and down phenomenon, asthma, cancer, congenital nystagmus, familial hemiplegic migraine, fetal movement disorder, and rheumatoid arthritis;

neurotic, stress-related and somatoform disorders such as social anxiety disorder, panic disorder, generalized anxiety disorder, obsessive compulsive disorder, post-traumatic stress disorder, and psychogenic movement disorder;

other degenerative diseases of basal ganglia such as pantothethenate kinase-associated neurodegeneration, progressive supranuclear palsy, multiple system atrophy, dyslexia, basal ganglion degeneration, and neuroferritinopathy;

other extrapyramidal and movement disorders such as demiballismus, extrapyramidal disorder, essential tremor, geniospasm, hyperexplexia, akathisia, ballismus/hemiballism, myoclonus, and restless legs syndrome/Willis-Ekbom's syndrome;

other nervous system or motor function such as sleep-related bruxism, abnormal involuntary movement disorders, alien limb syndrome, Alzheimer's disease (agitation), clumsiness, clonic hemifacial spasm, olfactory nerve agenesis, congenital cranial nerve paralysis, exercise ataxia syndrome, familial periodic paralysis, congenital hemiparesis, fine motor delay, fine motor skill dysfunction, gross motor delay, multiple sclerosis, congenital flaccid paralysis, congenital Horner's syndrome, alternating hemiplegia of childhood, motor developmental delay, cerebral palsy, athetoid cerebral palsy, posturing, pseudoparalysis, psychomotor hyperactivity, bradykinesia, synkinesis, akinesia, Riley-Day syndrome, and athetosis;

Parkinson's/parkinsonism such as parkinsonism, drug-induced parkinsonism, micrographia, and Parkinson's disease;

demoralization including demoralization and subjective incompetence

pediatric-onset behavioral and emotional disorders such as attention deficit hyperactivity disorder, attention deficit disorder, hyperkinesia, hyperkinesia (neonatal), oppositional defiant disorder, provisional tic disorder, persistent (chronic) motor or vocal tic disorder, stereotypic movement disorder, stereotypy, and Tourette's syndrome;

pervasive developmental disorders such as autism spectrum disorders, Rett's syndrome, Asperger's syndrome, pervasive developmental disorder NOS, and dyslexia; and

substance abuse or dependence such as addiction disorders, alcoholism, cocaine dependence, illegal drug abuse, methamphetamine abuse, methamphetamine addiction/dependence, methamphetamine use disorder, morphine abuse, morphine-analogue abuse, nicotine dependence, polysubstance abuse, and prescription drug abuse.

In some embodiments, the patient has been determined to have 22q11.2 deletion syndrome. In some embodiments, the patient is predisposed to developing a psychiatric disorder due to the patient having 22q11.2 deletion syndrome. In some embodiments, the patient has been determined to have COMT haploinsufficiency. In some embodiments, the patient is predisposed to developing a psychiatric disorder due to the patient having COMT haploinsufficiency.

In some embodiments, the patient has been determined to have Velocardiofacial syndrome (VCFS). In some embodiments, the patient with Velocardiofacial syndrome has a 3 Mb deletion. In some embodiments, the 3 Mb deletion comprises the deletion of COMT and TBX1. In some embodiments, the patient with Velocardiofacial syndrome has a 1.5 Mb deletion. In some embodiments, the 1.5 Mb deletion comprises the deletion of TBX1 and COMT.

Each and every method, composition, or use described herein also optionally includes the limitation “if the VMAT2 inhibitor is deutetrabenazine or tetrabenazine, then the VMAT2 inhibitor is administered to the patient to treat a neurological or psychiatric disease or disorder other than Huntington's Disease.

In some embodiments, the composition further comprises the step of monitoring the patient for one or more exposure-related adverse reactions. In some embodiments, the one or more exposure-related adverse reactions is chosen from hypersensitivity reactions. In some embodiments, the one or more exposure-related adverse reactions is chosen from hypersensitivity reactions with or without dermatological reactions. In some embodiments, the one or more exposure-related adverse reactions is chosen from hypersensitivity reactions with dermatological reactions. In some embodiments, the one or more exposure-related adverse reactions is chosen from hypersensitivity reactions without dermatological reactions. In some embodiments, the one or more exposure-related adverse reactions is chosen from allergic dermatitis, angioedema, pruritis, and urticaria.

In some embodiments, hypersensitivity is Type I hypersensitivity. In some embodiments, hypersensitivity is Type IV hypersensitivity.

In some embodiments, the one or more exposure-related adverse reactions is chosen from urticaria, pruritus, allergic dermatitis, and angioedema. In some embodiments, the one or more exposure-related adverse reactions is chosen from urticaria, allergic dermatitis, and angioedema. In some embodiments, the one or more exposure-related adverse reactions is hypersensitivity reaction and rash. In some embodiments, the one or more exposure-related adverse reactions is rash. In some embodiments, the one or more exposure-related adverse reactions is chosen from rash, urticaria, and reactions consistent with angioedema.

In some embodiments, the one or more exposure-related adverse reactions is chosen from reactions consistent with angioedema. In some embodiments, the one or more exposure-related adverse reactions that are consistent with angioedema are chosen from swelling of the face, lips, and mouth, and dyspnea.

In some embodiments, the patient in need thereof who is at increased risk of one or more exposure-related adverse reactions has a history of allergies. In some embodiments, the patient has a history of allergies to one or more drugs, e.g., penicillin or paroxetine; to one or more types of food, e.g., eggs, milk, peanuts, tree nuts, fish, shellfish, wheat or soy; and/or to cats. In some embodiments, the patient has a history of hives.

In some embodiments, the composition further comprises the step of administering to the patient that is experiencing one or more exposure-related adverse reactions one or more medications chosen from steroids and antihistamines. In some embodiments, the steroid is a systemic glucocorticoid, such as prednisone. In some embodiments, the steroid is a hydrocortisone cream. In some embodiments, the antihistamine is diphenhydramine.

In some embodiments, the patient is also being administered digoxin, and the method further comprises administering to the patient a therapeutically effective amount of the VMAT2 inhibitor. In some embodiments, the dose of digoxin is reduced.

In some embodiments, the patient is also being administered digoxin, and the method further comprises administering to the patient a therapeutically effective amount of the VMAT2 inhibitor, subsequently determining that the patient is to begin treatment with digoxin, and continuing administration of the therapeutically effective amount of the VMAT2 inhibitor to the patient. In some embodiments, the dose of digoxin is reduced.

In some embodiments, the patient is also being administered digoxin, and the method further comprises administering a therapeutically effective amount of the VMAT2 inhibitor to the patient, wherein the administration produces a mean digoxin Cₘₐₓ that is about 1.5 to 2.5 fold higher than the mean digoxin Cₘₐₓ for a patient who is administered digoxin alone and/or a mean digoxin AUC_{0-∞} that is about 1 to about 2 fold higher than the mean digoxin AUC_{0-∞} for a patient who is administered digoxin alone.

In some embodiments, the patient is a poor metabolizer of cytochrome P450 2D6 (CYP2D6). In certain embodiments, the patient has a CYP2D6 poor metabolizer genotype. In certain embodiments, the CYP2D6 poor metabolizer genotype is chosen from the CYP2D6G1846A genotype or the CYP2D6C100T genotype. In certain embodiments, the CYP2D6 poor metabolizer genotype is one of the CYP2D6G1846A (AA) genotype or the CYP2D6G1846A (AG) genotype. In certain embodiments, the CYP2D6 poor metabolizer genotype is the CYP2D6G1846A (AA) genotype. In certain embodiments, the CYP2D6 poor metabolizer genotype is one of the CYP2D6C100T (TT) genotype or the CYP2D6C100T (CT) genotype. In certain embodiments, the CYP2D6 poor metabolizer genotype is the CYP2D6C100T (TT) genotype. In some embodiments, the patient with CYP2D6 poor metabolizer genotype is administered an amount of about 40 mg of valbenazine free base once daily.

In some embodiments, administration to a patient who is a poor metabolizer of CYP2D6 results in increased exposure of (+)-α-3-isobutyl-9,10-dimethoxy-1,3,4,6,7,11b-hexahydro-2H-pyrido[2,1-a]isoquinolin-2-ol. In some embodiments, administration to a patient who is a poor metabolizer of CYP2D6 results in increased risk of one or more clinically significant parkinson-like signs or symptoms. In some embodiments, administration to a patient who is a poor metabolizer of CYP2D6 results in prolongation of the patient's QT interval. In certain embodiments, the therapeutically effective amount is less than the amount that is administered to a patient who is not a CYP2D6 poor metabolizer. In certain embodiments, the therapeutically effective amount is the same amount as that administered to a patient who is not a CYP2D6 poor metabolizer.

The choice of excipient, to a large extent, depends on factors, such as the particular mode of administration, the effect of the excipient on the solubility and stability of the active ingredient, and the nature of the dosage form.

The pharmaceutical compositions provided herein may be provided in unit dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions provided herein may be administered alone, or in combination with one or more other compounds provided herein, one or more other active ingredients. The pharmaceutical compositions provided herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art). The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### Oral Administration

The pharmaceutical compositions provided herein may be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pregelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Vee gum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-0-SIL® (Cabot Co. of Boston, Mass.); and mixtures thereof. The pharmaceutical compositions provided herein may contain about 0.1 to about 5% by weight of a lubricant. Suitable glidants include colloidal silicon dioxide, CAB-0-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation. The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or polyalkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems.

The pharmaceutical compositions provided herein may be provided as noneffervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide. Coloring and flavoring agents can be used in all of the above dosage forms. The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as antacids, proton pump inhibitors, and H2-receptor antagonists.

### Dosages

In the treatment, prevention, or amelioration of one or more symptoms of disorders or other conditions, disorders or diseases associated with VMAT2 inhibition, an appropriate dosage level generally is about 0.001 to 100 mg per kg patient body weight per day (mg/kg per day), about 0.01 to about 80 mg/kg per day, about 0.1 to about 50 mg/kg per day, about 0.5 to about 25 mg/kg per day, or about 1 to about 20 mg/kg per day, which may be administered in single or multiple doses. Within this range the dosage may be 0.005 to 0.05, 0.05 to 0.5, or 0.5 to 5.0, 1 to 15, 1 to 20, or 1 to 50 mg/kg per day. In certain embodiments, the dosage level is about 0.001 to 100 mg/kg per day.

In certain embodiments, the dosage level is about from 25 to 100 mg/kg per day. In certain embodiments, the dosage level is about 0.01 to about 40 mg/kg per day. In certain embodiments, the dosage level is about 0.1 to about 80 mg/kg per day. In certain embodiments, the dosage level is about 0.1 to about 50 mg/kg per day. In certain embodiments, the dosage level is about 0.1 to about 40 mg/kg per day. In certain embodiments, the dosage level is about 0.5 to about 80 mg/kg per day. In certain embodiments, the dosage level is about 0.5 to about 40 mg/kg per day. In certain embodiments, the dosage level is about 0.5 to about 25 mg/kg per day. In certain embodiments, the dosage level is about 1 to about 80 mg/kg per day. In certain embodiments, the dosage level is about 1 to about 75 mg/kg per day. In certain embodiments, the dosage level is about 1 to about 50 mg/kg per day. In certain embodiments, the dosage level is about 1 to about 40 mg/kg per day. In certain embodiments, the dosage level is about 1 to about 25 mg/kg per day.

In certain embodiments, the dosage level is about from 5.0 to 150 mg per day, and in certain embodiments from 10 to 100 mg per day. In certain embodiments, the dosage level is about 80 mg per day. In certain embodiments, the dosage level is about 40 mg per day.

For oral administration, the pharmaceutical compositions can be provided in the form of tablets containing 1.0 to 1,000 mg of the active ingredient, particularly about 1, about 5, about 10, about 15, about 20, about 25, about 30, about 40, about 45, about 50, about 75, about 80, about 100, about 150, about 200, about 250, about 300, about 400, about 500, about 600, about 750, about 800, about 900, and about 1,000 mg of the active ingredient for the symptomatic adjustment of the dosage to the patient to be treated. In certain embodiments, the pharmaceutical compositions can be provided in the form of tablets containing about 100 mg of the active ingredient. In certain embodiments, the pharmaceutical compositions can be provided in the form of tablets containing about 80 mg of the active ingredient. In certain embodiments, the pharmaceutical compositions can be provided in the form of tablets containing about 75 mg of the active ingredient. In certain embodiments, the pharmaceutical compositions can be provided in the form of tablets containing about 50 mg of the active ingredient. In certain embodiments, the pharmaceutical compositions can be provided in the form of tablets containing about 40 mg of the active ingredient. In certain embodiments, the pharmaceutical compositions can be provided in the form of tablets containing about 25 mg of the active ingredient. The compositions may be administered on a regimen of 1 to 4 times per day, including once, twice, three times, and four times per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds provided herein may also be combined or used in combination with other agents useful in the treatment, prevention, or amelioration of one or more symptoms of the diseases or conditions for which the compounds provided herein are useful, and other conditions commonly treated with antipsychotic medication.

In certain embodiments, the compounds provided herein may also be combined or used in combination with a typical antipsychotic drug. In certain embodiments, the typical antipsychotic drug is fluphenazine, haloperidol, loxapine, molindone, perphenazine, pimozide, sulpiride, thioridazine, or trifluoperazine. In certain embodiments, the antipsychotic drug is an atypical antipsychotic drug. In certain embodiments, the atypical antipsychotic drug is aripiprazole, asenapine, clozapine, iloperidone, olanzapine, paliperidone, quetiapine, risperidone, or ziprasidone. In certain embodiments, the atypical antipsychotic drug is clozapine.

Such other agents, or drugs, may be administered, by a route and in an amount commonly used thereof, simultaneously or sequentially with the compounds provided herein. When compounds provided herein are used contemporaneously with one or more other drugs, a pharmaceutical composition containing such other drugs in addition to the compounds provided herein may be utilized, but is not required. Accordingly, the pharmaceutical compositions provided herein include those that also contain one or more other active ingredients or therapeutic agents, in addition to the compounds provided herein.

The weight ratio of the compounds provided herein to the second active ingredient may be varied, and will depend upon the effective dose of each ingredient. Generally, an effective dose of each will be used. Thus, for example, when the compounds provided herein are used in combination with the second drug, or a pharmaceutical composition containing such other drug, the weight ratio of the particulates to the second drug may range from about 1,000:1 to about 1:1,000, or about 200:1 to about 1:200.

Combinations of the particulates provided herein and other active ingredients will generally also be within the aforementioned range, but in each case, an effective dose of each active ingredient should be used.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### Example 1

1. Estimated Exposure

Patient exposure was estimated based on dispensation of INGREZZA through specialty pharmacies, the primary route of INGREZZA prescription fulfillment. Estimated total exposure in patient-months was calculated based on the number of patients who were dispensed INGREZZA and the number of days' supply each patient received through 10 Apr. 2018. It was assumed that all patients who were dispensed INGREZZA took the drug according to labeled directions for use. Since the approval of INGREZZA (11 Apr. 2017) through 10 Apr. 2018, a total of approximately 10,269 patients received at least 1 shipment of INGREZZA with an estimated exposure of approximately 31,248 patient months.

2. Methodology

This safety analysis includes an evaluation of all relevant postmarketing cases from the Sponsor's safety database. The Sponsor's safety database includes both spontaneous and solicited reports. Reports received from the Sponsor's Patient Support Program and specialty pharmacies are considered solicited sources.

### 2.1. Postmarketing Adverse Reaction Analysis Methodology

A standardized Medical Dictionary for Regulatory Activities (MedDRA) query (SMQ, MedDRA version 20.1) for Parkinson-like Events (broad) was used to identify cases with SAEs mapping to the preferred terms (PTs) of this SMQ. The search period was from 11 Apr. 2017 (INGREZZA approval date) through 10 May 2018.

3. Data Presentation

### 3.1. Postmarketing Data

A total of 19 case reports with 1 or more PTs in the search criteria were identified in the postmarketing safety database. These 19 case reports underwent further medical review and were categorized based on Sponsor causality assessment of the Parkinson-like events. In 10 (7 solicited, 3 spontaneous) out of the 19 cases, at least 1 of the reported Parkinsonlike events were assessed as possibly related to INGREZZA.

In the remaining 9 (7 solicited, 2 spontaneous) case reports, the Parkinson-like events were assessed as having insufficient evidence to establish a causal relationship to INGREZZA. In several cases, alternative explanations for the Parkinson-like events were reported. The event Motor dysfunction in Case 2017NBI01466 occurred after anti-Parkinson medication was discontinued at the same time that INGREZZA was started. Tremor in Case 2018NBI01364 occurred in a patient with pre-existing tremor. In Case 2018NBI01365, Tremor was explained by concomitant anxiety, and in Case 2017NBI00717, Tremor was explained by a history of Parkinson's disease. Delayed onset of events after starting INGREZZA (time to onset >3 months) in Cases 2017NBI01590 and 2018NBI00975 made a causal relationship less likely. Parkinson-like events were reported after patients took a higher dose than recommended in the USPI in Case 2017NBI01732 and possibly in Case 2018NBI00674.

In the Phase 2 and 3 controlled studies in adults with tardive dyskinesia (treatment up to 6 weeks), the incidence of Parkinson-like adverse events (AEs) was 3% (vs. <1% for placebo) and 4% during long-term treatment with valbenazine (up to 48 weeks). No serious Parkinson-like AEs were reported from NBI clinical studies in tardive dyskinesia patients.

Of the 19 postmarketing cases with relevant SAEs presented in this report, 10 cases reported serious Parkinson-like events that were assessed as possibly related to INGREZZA by the Sponsor and underwent detailed review. The events, 7 of which were solicited, were commonly described as the patient being “frozen,” “barely able to move,” “unable to walk,” “having unsteady gait,” or “worsening tremor.” The events led to hospitalization in 6 of the cases and the other 4 events were assessed as importantmedical events. The outcome was reported as resolved or resolving in 7 cases, and unresolved for 1 case. No information about the outcome was provided in the remaining 2 cases.

In view of the information reported in these 10 cases, there is a possible causal relationship between the events of parkinsonism and INGREZZA. The possible causal relationship is supported by:
a plausible temporal relationship; time from starting INGREZZA to onset of the events ranged from 1 day to 2 months (within 2 weeks from starting INGREZZA in most cases);
positive de-challenge was reported in several cases (Case 2017NBI00265, 2017NBI00642, 2018NBI00355, and 2018NBI00781);
the drug's pharmacological action of reduction of dopamine release through reversible inhibition of presynaptic human vesicular monoamine transporter type 2.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-100. (canceled)

101. A method of treating a patient with tardive dyskinesia, comprising:
orally administering a therapeutically effective amount of a vesicular monoamine transporter 2 (VMAT2) inhibitor to the patient, wherein the VMAT2 inhibitor is (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester ditosylate, and wherein the therapeutically effective amount is an amount equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily for one week, and 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily after one week;
monitoring the patient for the presence or absence of one or more clinically significant parkinson-like signs or symptoms;
if the patient experiences one or more clinically significant parkinson-like signs or symptoms within the first two weeks after starting or increasing the dose of the VMAT2 inhibitor, administering a reduced amount or discontinuing the administration of the VMAT2 inhibitor to the patient; and
if the patient does not experience one or more clinically significant parkinson-like signs or symptoms within the first two weeks after starting or increasing the dose of the VMAT2 inhibitor, continuing administering the amount equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily to the patient.

102. The method of claim 101, wherein the patient experiences one or more clinically significant parkinson-like signs or symptoms within the first two weeks after starting or increasing the dose of the VMAT2 inhibitor, and the administration of the VMAT2 inhibitor to the patient is discontinued.

103. The method of claim 101, wherein the patient experiences one or more clinically significant parkinson-like signs or symptoms within the first two weeks after starting or increasing the dose of the VMAT2 inhibitor, and a reduced amount of the VMAT2 inhibitor is administered to the patient.

104. The method of claim 103, wherein the reduced amount is an amount equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

105. The method of claim 103, wherein the reduced amount is an amount equivalent to about 60 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

106. The method of claim 101, wherein the VMAT2 inhibitor is administered in the form of a tablet or capsule.

107. The method of claim 101, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester ditosylate is in polymorphic Form I.

108. The method of claim 101, wherein the one or more clinically significant parkinson-like signs or symptoms is chosen from falls, gait disturbances, tremor, drooling, and hypokinesia.

109. The method of claim 101, wherein the one or more clinically significant Parkinson-like signs or symptoms is chosen from difficulty moving or loss of ability to move muscles voluntarily, tremor, gait disturbances, and drooling.

110. The method of claim 101, wherein the patient does not experience one or more clinically significant parkinson-like signs or symptoms within the first two weeks after starting or increasing the dose of the VMAT2 inhibitor, and the VMAT2 inhibitor is continuously administered in the amount equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily to the patient after the first two weeks.

111. The method of claim 101, wherein the one or more clinically significant Parkinson-like signs or symptoms is tremor.

112. The method of claim 101, wherein the one or more clinically significant Parkinson-like signs or symptoms is drooling.
